(19) United States
(12) Patent Application Publication
(10) Pub. No.: US 2022/0319650 A1
(43) Pub. Date: Oct. 6, 2022

(54) Method and System for Providing Information About a State of Health of a Patient

(71) Applicant: Siemens Healthcare GmbH, Erlangen (DE)

(72) Inventors: Svenja Lippok, Uttenreuth (DE); Sven Kohle, Erlangen (DE); Stefan Thesen, Dormitz (DE); Volker Schaller, Uttenreuth (DE); Felix Nensa, Essen (DE)

(21) Appl. No.: 17/708,174

(22) Filed: Mar. 30, 2022

(30) Foreign Application Priority Data
Mar. 30, 2021 (DE) 10 2021 203 234.6

(51) Int. Cl. G16H 10/60; G16H 50/30; G16H 50/20; G16H 50/70

(52) U.S. Cl. CPC G16H 10/60; G16H 50/30; G16H 50/20; G16H 50/70

(57) ABSTRACT

A computer-implemented method for providing information about a state of health of a patient may include: receiving a plurality of patient information of the patient by means of an interface, ascertaining health information about the patient by means of a computing unit as a function of the plurality of patient information and a first function, and checking whether a trigger condition is fulfilled based on the ascertained health information about the patient, as well as providing the ascertained health information about the patient as a function of the trigger condition.

### CROSS REFERENCE TO RELATED APPLICATIONS

This patent application claims priority to German Patent Application No. 10 2021 203 234.6, filed Mar. 30, 2021, which is incorporated herein by reference in its entirety.

### BACKGROUND

### Field

The disclosure relates to a computer-implemented method and a system for providing information about a state of health of a patient.

### Related Art

Medical practitioners from many specialist fields are responsible for monitoring patients as well as for performing diagnostic examinations over a period of treatment of the patients. Examples of this include performing oncological follow-up imaging scans and keeping various types of patient information under observation, such as e.g. symptoms, blood values, tumor markers, as well as measured values which are associated with a physical constitution of the patient (e.g. weight, pulse, body fat percentage). In such cases, the medical practitioners must identify and interpret a change in patient information and, given an appropriate indication, order a suitable diagnostic examination. At the same time, it is important to assess patient information holistically, i.e. in context with other patient information. This may represent a difficulty in particular for consulting physicians since the exact state of the patient is often known only to a ward physician or primary care physician.

Furthermore, individual types of patient information are often distributed over a plurality of databases, such as e.g. medical information systems and different medical devices. Due to incompatibilities between the information systems and medical devices and/or limited access rights, accessing different pieces of patient information may prove difficult in practice and is very complicated and time-consuming for the medical practitioner. In particular patient information in written form, such as e.g. diagnostic findings or descriptions of symptoms, may generate a high overhead in terms of its acquisition and interpretation and is difficult and complicated to interpret in the context of other types of patient information, such as e.g. blood values or diagnostic images. Moreover, a plurality of patient information is nowadays acquired by means of private mobile devices, such as e.g. smartphones, smartwatches and/or tablets. This patient information can make a significant contribution to an assessment of the state of health of the patient but is often not accessible to the medical practitioner.

Already today, specialized applications (e.g. syngo.via) enable individual patient information to be detected, quantified and visualized. There are also already solutions which provide a predetermined subset of interrelated patient information in dedicated decision support applications (e.g. AI Pathway Companion) on the basis of guidelines defined by expert communities. In such applications, however, the patient must be included in a clinical aftercare program for a particular medical condition. At present, therefore, a proactive monitoring of the state of health of the patient is only available in dedicated programs for specific medical conditions. Furthermore, patient information for which a direct relationship with the specific medical condition has not yet been established is not considered in such approaches.

### BRIEF DESCRIPTION OF THE DRAWINGS/FIGURES

The accompanying drawings, which are incorporated herein and form a part of the specification, illustrate the embodiments of the present disclosure and, together with the description, further serve to explain the principles of the embodiments and to enable a person skilled in the pertinent art to make and use the embodiments.

FIG. 1 shows a schematic view of a system according to an exemplary embodiment of the disclosure.

FIG. 2 shows a schematic view of a system according to an exemplary embodiment of the disclosure.

FIG. 3 shows a schematic view of a training system according to an exemplary embodiment of the disclosure.

FIG. 4 shows a schematic view of a training system according to an exemplary embodiment of the disclosure.

FIG. 5 shows a flowchart of a method according to an exemplary embodiment of the disclosure.

FIG. 6 shows a flowchart of a method according to an exemplary embodiment of the disclosure.

FIG. 7 shows a flowchart of a method according to an exemplary embodiment of the disclosure.

The exemplary embodiments of the present disclosure will be described with reference to the accompanying drawings. Elements, features and components that are identical, functionally identical and have the same effect are—insofar as is not stated otherwise—respectively provided with the same reference character.

### DETAILED DESCRIPTION

In the following description, numerous specific details are set forth in order to provide a thorough understanding of the embodiments of the present disclosure. However, it will be apparent to those skilled in the art that the embodiments, including structures, systems, and methods, may be practiced without these specific details. The description and representation herein are the common means used by those experienced or skilled in the art to most effectively convey the substance of their work to others skilled in the art. In other instances, well-known methods, procedures, components, and circuitry have not been described in detail to avoid unnecessarily obscuring embodiments of the disclosure. The connections shown in the figures between functional units or other elements can also be implemented as indirect connections, wherein a connection can be wireless or wired. Functional units can be implemented as hardware, software or a combination of hardware and software.

An object of the present disclosure is to provide information about a state of health of a patient as a function of a plurality of patient information which is selected independently of a medical condition of the patient. It is furthermore an object of the present disclosure to provide information about a state of health of a patient based on patient information about the patient that is available also outside of a specific issue and/or case configuration, and thereby enable the state of health of the patient to be monitored.

According to one aspect, a computer-implemented method is provided for controlling a diagnostic assessment station in a medical information network comprising a computing unit and at least one diagnostic assessment station that maintains a data connection to the computing unit and is intended for producing medical findings for a patient by a user. The method comprises a number of steps. One step is directed to a receiving of a plurality of patient information of the patient at the computing unit by means of an interface, the patient information containing at least two different medical parameters assigned to the patient. A further step is directed to an ascertaining of health information about the patient by applying a first function hosted in the computing unit to the patient information by means of the computing unit. A further step is directed to a checking by the computing unit whether a trigger condition is fulfilled based on the ascertained health information. A further step is directed to a providing of control commands for controlling the diagnostic assessment station by means of the computing unit based on the trigger condition, the control commands being suitable for prioritizing the patient in a worklist of the user hosted in the diagnostic assessment station as a function of the ascertained health information, and/or to output the ascertained health information about the patient to the user via the diagnostic assessment station. A further step is directed to an outputting of the control commands to the diagnostic assessment station by means of the computing unit.

According to a further aspect, a method of providing information about a state of health of a patient is provided which comprises the following steps:
receiving a plurality of patient information of the patient by means of an interface, wherein the plurality of patient information includes at least two different medical parameters assigned to the patient,
ascertaining health information about the patient by means of a computing unit as a function of the plurality of patient information and a first function and checking whether, based on the ascertained health information about the patient, a trigger condition is fulfilled,
providing the ascertained health information about the patient as a function of the trigger condition, wherein the providing comprises
prioritizing the patient in a worklist of a user as a function of the ascertained health information about the patient, and/or
outputting the ascertained health information about the patient to the user, and/or
storing the ascertained health information about the patient in a memory unit.

The methods are in particular a computer-implemented method. This may mean that the method according to the disclosure is coordinated and carried out by means of a computing unit of a local computer, of a network computer, of a server, of a cloud or of a comparable component. The performance of the method may be initiated for example as a result of a manual activation by a user. In an exemplary embodiment, the method is started automatically, e.g. on account of receiving patient information or after the arrival of a predetermined criterion. A predetermined criterion may for example represent an agreement between patient information and a selection criterion of a clinical trial, a description of symptoms of the patient, an elapsing of a predetermined period of time, a predetermined frequency of the monitoring of the state of health, an admission of a new patient or the like. Any other criteria are, of course, also conceivable.

The medical network or medical information network may be configured for exchanging medical information, i.e. in particular patient information and/or health information and the like. The medical information network may also be configured to exchange control commands between the connected components. The medical network may in particular establish a communications link and/or data connection between the diagnostic assessment station and the computing unit. In addition, the medical network may establish a communications link and/or data connection to further medical data processing devices such as, say, storage devices for storing patient information. The medical network may comprise an intranet and/or an internet. In other words, the diagnostic assessment station may maintain a communications link and/or data connection to the computing unit via the internet.

According to embodiments, communications links and/or data connections may be based on the HL7 standard. Health Level 7 (HL7 ) is a set of international standards for exchanging data between healthcare organizations and their computer systems. In particular, communications links and/or data connections may be based on the FHIR standard. Fast Healthcare Interoperability Resources (FHIR) is a standard developed by HL7 . It supports data exchange between software systems in the healthcare sector. By using the HL7 or FHIR standard it is possible to transfer data in a structured format with no need for reformatting.

The diagnostic assessment station may be configured in particular as a front-end computing device at which a user (in particular a member of the medical staff such as a doctor) may retrieve and/or view and/or analyze patient information, and/or at which the user may retrieve and/or view and/or modify medical reports. In particular, the diagnostic assessment station may be configured in such a way that it hosts a worklist for the user. The worklist may include a list of patients that the user is to diagnostically assess at the diagnostic assessment station. In particular, the worklist may specify an order or a prioritization in which the user is to diagnostically assess the listed patients. The diagnostic assessment station may include a user interface. The diagnostic assessment station may be configured as a device known as a client.

The computing unit may be configured as a back-end computing device and in particular as a server system. The computing unit may comprise a cluster or a group of computing devices and data storage facilities. The computing unit may have no user interface for the user (of the diagnostic assessment station). The computing unit may maintain a data connection to the front-end computing device via the medical network. The computing unit may maintain a data connection to a number of different (but in particular similar) diagnostic assessment stations via the medical network. The diagnostic assessment station(s) may belong to a medical organization, such as, for instance, a practice, a hospital or a hospital network. The computing unit may also belong to the medical organization or be configured outside of the medical organization. The computing unit may maintain a data connection via the medical network to a number of different diagnostic assessment stations, each belonging to different medical organizations.

The state of health of the patient may be characterized by the plurality of patient information of the patient. In particular, the state of health of the patient may be characterized by one or more medical parameters contained in the patient information and/or by one or more variables derived from the one or more medical parameters. The multiple medical parameters may in this case represent in particular a parameter configuration or a pattern of parameters which possess a functional and/or an indicative relationship with a medical history, a medical condition, an impairment, a lifestyle and/or an influencing of the patient by external circumstances. It is furthermore conceivable that the state of health of the patient is characterized by a correlation of one or more medical parameters and/or one or more derived variables. Furthermore, the state of health may also be characterized by a correlation of one or more medical parameters and/or derived variables of the patient with one or more corresponding medical parameters and/or derived variables of a reference patient. A correlation may in this case comprise in particular a comparison and/or a quantification of a deviation of one or more medical parameters and/or derived variables of the patient with regard to one or more corresponding medical parameters and/or derived variables of a reference patient.

The plurality of patient information in this case includes at least two different medical parameters assigned to the patient. In particular, a medical parameter may comprise one or more numeric or measured values and/or a temporal sequence of a numeric or measured value. A medical parameter may be for example a blood value, a urine value, a pulse value, a blood oxygen saturation, a sleep behavior, a tumor marker, a weight, a body height, an age, a gender or the like. However, it is equally conceivable that the medical parameter comprises an arbitrary measured value of a diagnostic medical device. For example, the medical parameter may relate to a cardiac activity of an electrocardiogram, a measured value of a molecular diagnostic analysis, or even a result of a histological examination. Also, to be understood by the term medical parameter is information about internal and external structures of the patient, which may be derived e.g. on the basis of imaging methods such as magnetic resonance tomography, computed tomography, various X-ray methods, ultrasound, positron-emission tomography or the like. For example, medical parameters may comprise organ volumes, such as, say, a heart or lung volume. Further, medical parameters may comprise a location, a number and/or a size of changes, in particular pathological changes, in the body of the patient. Said pathological changes may comprise e.g. lesions or nodes in tissue regions of the patient. Further, the medical parameter may comprise one or more pieces of semantic information. In particular, medical parameters may comprise one or more words. In particular, one or more of the medical parameters may represent a diagnosis and/or a description of a symptom in a diagnostic finding.

A variable derived from the one or more medical parameters may be based for example on a (mathematical) derivative of one or more of the medical parameters, a mean value, a variance, a mathematical integration of one or more of the medical parameters, a correlation of one or more of the medical parameters, a superordinate semantic concept, etc.

In an exemplary embodiment, the plurality of patient information is selected independently of a medical condition of the patient. This can mean that the medical parameters of the plurality of patient information are uncorrelated with a specific medical condition and/or a medical issue. In an exemplary embodiment, the plurality of patient information includes a high percentage, e.g. more than 60%, more than 70% or more than 80%, of medical parameters which can be acquired within the scope of a routine examination, such as e.g. a blood pressure measurement, a weight measurement, a dialog with the patient and/or a physical inspection of the patient. The plurality of patient information may therefore be understood in particular as undirected with regard to a specific medical indication and/or a medical condition and/or a medical issue of the patient.

The receiving of the plurality of patient information may comprise at least a receiving of first patient information and second patient information from a first information source. The first patient information may in this case comprise a first medical parameter of the patient, while the second patient information comprises a second medical parameter of the patient. An information source may comprise e.g. a memory unit, a medical device, a private device of the patient and/or a medical information system. The plurality of patient information may be received via an arbitrary interface or a plurality of arbitrary interfaces. For example, the receiving of the plurality of patient information may comprise an establishment of a communications link to a database, a medical information system, a medical device, a cloud, a server and/or a private device of a patient. The plurality of patient information may be transmitted to the computing unit by means of the established communications link. The communications link may in this case comprise a wired or wireless connection.

The health information about the patient may represent a selection of medical parameters which characterize a physical constitution of the patient. A selection of medical parameters may for example comprise a parameter configuration or a pattern of medical parameters of the patient. The health information may furthermore comprise a parameter configuration which points to a change in the state of health of the patient. It is equally conceivable that the health information points to a likelihood of a medical condition and/or an, in particular critical, change in the state of health of the patient. The health information may in this case include in particular a combined or aggregated representation of selected medical parameters which contribute toward a change in the physical constitution of the patient. It is further conceivable that the health information comprises one or more key indicators which are derived as a function of one or more medical parameters and quantify a change in the physical constitution of the patient.

In an exemplary embodiment, the first function is configured to ascertain the health information about the patient based on the plurality of patient information. For this purpose, the method according to the disclosure may comprise in particular a step of applying the first function to the plurality of patient information. However, it is equally conceivable that the plurality of patient information is input into the first function in one step.

A prioritizing of the patient in the worklist may comprise in particular that the patient is allocated a place in the worklist or that the patient is ranked into the order of the worklist. In this case the patient may already have a place in the worklist, which is then updated. Alternatively, the patient may be newly added to the worklist in accordance with the prioritization.

As a result of the automated analysis of different, in particular orthogonal, pieces of patient information and the provision of the corresponding health information or the prioritizing of the patient in the worklist, the user is automatically furnished with information with which he or she is able to judge the relevance of the respective case without having personally to study the patient information. The user can thus be made aware of hidden problems of the patient and can respond accordingly.

According to one aspect, the first function is configured to detect multivariate outliers in patient information.

Multivariate outlier detection is an established concept. In the present application case, multivariate outliers may denote isolated data points in the patient information which, taken in their totality, indicate a state of health of and in particular a health risk to the patient. In contrast to univariate outliers, such as, say, a dangerously high blood pressure, which in isolation indicate a state of health and in particular a health risk and should be detected by conventional data evaluations, multivariate outliers are difficult to detect. For example, patient information containing e.g. normal values, taken individually, for body mass index, blood sugar level, a specific lesion growth in the lung, etc., when considered in combination, nonetheless points to a medical problem. The problem becomes all the more serious, the higher-dimensional the parameter space is. The more parameters it is necessary to monitor, the more difficult it is to detect anomalous data constellations. By using a multivariate outlier detection function it is possible to detect such constellations automatically.

In exemplary embodiments, multivariate outliers may comprise multiple individual values from the patient information which, taken in their totality, indicate a state of health of and in particular a health risk to the patient. Accordingly, the first function for detecting multivariate outliers may be configured to extract a plurality of different values from the patient data and to check whether these, in combination, indicate a multivariate abnormality in the patient information which indicates a state of health of and in particular a health risk to the patient.

In other words, multivariate outlier detection may be used to identify possible abnormalities in the patient information which may represent in particular a health risk to the patient. The abnormalities or health risks may be provided as health information on the basis of which e.g. a prioritization may then be conducted.

According to one aspect, the first function for detecting multivariate outliers comprises a trained function and in particular implements one or more of the following algorithms:
isolation forest,
elliptic envelope,
fast-minimum covariance determinant estimator (Fast MCD), and/or
local outlier factors (LOF).

A trained function generally maps input data to output data. In this case the output data may be dependent in particular on one or more parameters of the trained function. The one or more parameters of the trained function may be determined and/or adjusted by means of a training process. The determination and/or adjustment of the one or more parameters of the trained function may be based in particular on a pair composed of training input data and associated training output data, the trained function being applied to the training input data in order to generate training imaging data. In particular, the determination and/or adjustment may be based on a comparison of the training imaging data and the training output data. Generally, a trainable function, i.e. a function having parameters that have not yet been adjusted, is also referred to as a trained function. In particular, the trained function may be contained in a single filter component of the data filter. In addition, the data filter may also contain further filter components which comprise no trained functions because they operate e.g. according to a rule-based principle. Furthermore, the data filter may also include a number of trained functions.

Other terms for trained function are trained mapping rule, mapping rule having trained parameters, function having trained parameters, artificial-intelligence-based algorithm, machine learning algorithm. An example of a trained function is an artificial neural network.

As a result of the use of a trained function, the first function may also be adapted to highly complex patient information comprising multidimensional parameter sets.

The cited algorithms, isolation forest, elliptic envelope, fast-minimum covariance determinant estimator (Fast MCD), and/or local outlier factors (LOF), have proved their worth as suitable algorithms for multivariate outlier detection for other applications outside the field of health informatics (cf. “Isolation forest”, Liu FT et al., 8th IEEE international conference on data mining, 2008; “A fast algorithm for the minimum covariance determinant estimator” Rousseeuw P J, Technometrics 1999; 41(3): 212-23; “LOF: identifying density-based local outliers”, Breunig M M et al., in Proc. ACM SIGMOD 2000). The inventors have recognized that these algorithms for problem formulation in the medical information network permit a good characterization of a state of health and in particular are able to detect hidden health risks in patient information.

According to one aspect, the first function is further configured to determine an abnormality value for at least a part of the patient information, wherein, in the step of checking the trigger condition, the trigger condition is fulfilled when the abnormality value exceeds a predefined threshold.

The abnormality value may in this case specify the degree to which the patient information deviates from an, in particular predefinable, norm and/or a normal value. An abnormality value may be an arbitrary value, in particular a numeric value, which specifies a degree to which a part of the patient information deviates from a norm. The norm may relate e.g. to empirical values (learned values), values of a patient cohort or a part of the patient information that is different from the at least one part. In particular, the different part may relate to a different time point in the patient history than the part of the patient information for which the abnormality value is provided. In particular, the abnormality value may be output by a trained function contained in the first function (or, as the case may be, a trained function can be adapted to that effect).

In particular, the abnormality value may be defined such that it is all the higher, the greater a health risk is to a patient.

In particular, the norm may be predefined. In particular, the norm may be predefined specifically for the patient. In particular, the norm may be predefined. In particular, the norm may be adaptively predefined specifically for the patient. In particular, the norm may be predefined specifically for the patient based on the patient information by the correspondingly embodied first function. In particular, the norm may be predefined specifically for the patient based on the patient information by a trained function contained in the first function.

As a result of the calculation of an abnormality value, the state of health becomes comparable in particular across a plurality of patients, which permits a better comprehensibility for the user and a simpler prioritization. According to one aspect, the health information is ascertained based on the abnormality value and/or the health information comprises the abnormality value.

According to one aspect, the control commands are suitable for prioritizing the patient in the worklist as a function of the abnormality value, in particular the patient being prioritized all the higher, the higher the abnormality value is.

According to one aspect, the step of ascertaining the health information comprises determining a number of different abnormality values for the patient information by applying a number of different first multivariate outlier detection functions to the patient information by means of the computing unit, the health information being based on: an aggregated abnormality value from the different abnormality values, and/or an average abnormality value from the different abnormality values.

Thanks to the use of different functions, in particular orthogonal abnormality values may be determined, which in particular may indicate how different parts of the patient information deviate in each case from a norm and/or a normal value. An accurate picture of the deviation may be determined as a result.

In particular, a part of the patient information may in this case relate to medical image data, while e.g. another relates to laboratory data.

According to one aspect, the method further comprises the following steps:
providing patient information of a plurality of comparison patients in each case, each comparison patient being associated with previously known health information, and
determining one or more reference patients from a plurality of comparison patients based on similarity measures, a similarity measure being based on a similarity between the patient information of the patient and the patient information of the comparison patients, and
wherein, in the step of ascertaining the health information, the health information is ascertained in addition based on the previously known health information of the reference patients.

In other words, in order to improve the provided health information, it is provided to conduct a search by means of automatic processing, on the basis of the patient information of the present patient, for similar patients for whom already established health information is (previously) known and, optionally, has been verified. This is based on the idea that findings from similar cases may potentially be relevant to the present case. To that end, it is provided to identify reference patients in a set of comparison patients, which reference patients display a certain similarity to the present patient. For this purpose, the patient information of the present patient is compared with the patient information of each of the comparison patients. The patient information of the comparison patients may have a similar structure and content to the patient information of the present patient. The patient information data of the comparison patients may be stored in one or more databases which may be, as it were, part of the medical information network.

In order to determine the reference patients, all the available patient information of the comparison patients may be analyzed to assess its similarity to the patient information of the present patient. A similarity measure may be determined in each case for the comparison patients based on a similarity between the patient information of the respective comparison patient and the present patient and in particular indicating or quantifying a similarity. A similarity measure may for example be a numeric value or “score”. The similarity measures may be determined for example based on the application of a similarity metric which outputs a similarity measure based on the input variables, i.e. the patient information. The similarity metric may in this case be implemented in particular in a data processing algorithm (of a third function) which e.g. is likewise hosted in the computing unit. Reference patients are in particular such comparison patients that, based on the respective patient information data, reveal a certain similarity to the present patient. In other words, reference patients may in particular be such comparison patients whose similarity measure referred to the patient information data exceeds a predetermined or predefined or predefinable threshold.

Since each comparison patient is associated with at least one piece of health information, the automatic search for similar patients returns a selection of health information that is potentially relevant to the present patient.

According to a development, determining the one or more reference patients comprises the steps of:
extracting a data descriptor from the patient information of the present patient,
receiving a corresponding data descriptor in each case for each of the comparison patients,
determining a similarity measure for each comparison patient, a similarity measure being based in each case on a similarity between the data descriptor and a corresponding data descriptor, and
determining the one or more reference patients based on the determined similarity measures.

The data descriptor may include one or more features that have been extracted from the patient information or calculated therefrom. Another name for data descriptor may be the term “feature signature”. The data descriptor may in particular characterize the patient information. The features of the data descriptor may be combined to form a feature vector. In particular, the data descriptor may contain such a feature vector. Features extracted from image data may be morphological and/or structural features and/or features relating to a texture and/or to a pattern. Features extracted from non-image data may be features relating to findings, a medical report, a measured value, demographic information, etc. The computing unit may be configured in particular to determine similarity measures based on the data descriptor or to host a corresponding data processing algorithm.

Determining the similarity measures may comprise extracting or receiving a corresponding data descriptor in each case from the patient information of the comparison patients. Determining the similarity measures may further comprise comparing the corresponding data descriptors with the data descriptor in each case. The step of comparing may be based in particular on the determining of a distance of the respective data descriptors in the feature space, the calculation of a cosine similarity of the data descriptors and/or the calculation of a weighted sum of the difference or similarity of individual features of the data descriptor. In particular those comparison patients may be identified as reference patients whose associated similarity measure is greater than a predefined or predefinable threshold.

By using data descriptors, easy-to-implement and readily transferable parameters are defined for synchronizing different patient information data. Furthermore, the features contained in the feature signatures may be based on superordinate observables derived from the datasets, which observables often characterize the properties of the datasets better than the underlying data itself.

According to one aspect, determining the one or more reference patients comprises applying a trained function in each case to the patient information of the present patient and the comparison patients, which trained function is configured to determine a similarity measure between patient information data or, as the case may be, to extract data descriptors from patient information data and determine a similarity measure between patient information on the basis of the extracted data descriptors.

According to one aspect, the patient information comprises at least one medical image dataset and the health information is based on a measured value to be extracted from the medical image dataset, wherein the step of ascertaining health information comprises applying an image processing algorithm in order to extract the measured value from the medical image dataset. In particular, the image processing algorithm may be hosted by the computing unit.

According to one aspect, the measured value to be extracted comprises a dimension of a lesion in a part of the patient's body imaged in the image dataset and the image processing algorithm is configured to detect and/or quantify lesions in medical image datasets.

According to one aspect, the medical network further comprises an examination modality for performing a medical examination on the patient and/or a planning unit for planning a medical examination on the patient. Further, the method comprises a step of determining, by means of the computing unit, a medical examination to be performed for the patient, and a step of providing second control commands by means of the computing unit, the second control commands being suitable for controlling the examination modality and/or the planning unit in such a way that the medical examination to be performed is at least reserved and/or initiated. In particular, the method may further comprise a step of transmitting the second control commands and the examination modality and/or the planning unit. In particular, the examination modality may comprise an imaging modality. In particular, the second control commands may comprise examination parameters on the basis of which the examination can be performed. Examination parameters may comprise e.g. scanning protocols (e.g. MR sequences) and/or settings of an imaging modality, etc.

As a result of the above aspect, examinations of the patient may be initiated automatically based on the health information, which further reduces the user workload.

According to one aspect, the step of ascertaining the health information comprises determining a parameter configuration from the plurality of patient information, and ascertaining the health information based on the determined parameter configuration.

A parameter configuration may in particular comprise a selection of medical parameters from the plurality of patient information of the patient. The selection of medical parameters may in particular reveal a causal relationship with a health history, a medical condition, an impairment, a lifestyle and/or an influencing of the patient by an external circumstance. This may mean that the selected medical parameters have a direct or indirect impact on the state of health of the patient and/or result as a consequence of a behavior, a state of health and/or an external factor acting on the patient.

According to one aspect, the parameter configuration comprises:
a selection of a number of individual medical parameters which in their totality characterize a physical state of the patient and/or point to a change in a state of health of the patient, and/or one or more key indicators derived as a function of one or more medical parameters which characterize a physical state of the patient and/or point to a change in a state of health of the patient.

The parameter configuration may in particular reveal a functional relationship with the health history, the medical condition, the impairment, the lifestyle and/or the influencing of the patient by an external circumstance. This may mean that the health history, the medical condition and/or the impairment of the patient can be quantified on the basis of the health information and/or the parameter configuration and/or that the lifestyle of the patient and/or an external circumstance change the state of health in a quantifiable manner The parameter configuration may, of course, equally reveal an indicative relationship with the health history, the medical condition, the impairment, the lifestyle and/or the influencing of the patient by an external circumstance. An indicative relationship may in this context be understood as a presumed and/or a purely qualitatively determinable dependence.

In an exemplary embodiment, the first function is configured to extract the parameter configuration from the plurality of patient information of the patient. The plurality of patient information may in this case include in particular information about a behavior, such as e.g. a sleeping behavior, an eating behavior, a movement behavior, a hygiene behavior or the like. Such patient information may be received for example from a smart device of the patient.

By determining the parameter configuration from the plurality of patient information it is advantageously possible to map a physical constitution of the patient, but also a change in the physical constitution of the patient, as a function of a behavior, an external circumstance and/or a medical condition. In this case it is also possible to consider influences which are unknown to a treating physician or usually are not linked to a specific state of health.

The first function may be configured in particular to extract one or more medical parameters and, optionally, to determine one or more derived variables from the medical parameters. It is further conceivable that the first function is configured to determine the health information about the patient as a function of one or more medical parameters and/or derived variables. For this purpose, the first function may in particular be configured to carry out a classification of one or more medical parameters. In addition, the first function may be configured to correlate and/or compare patient information, in particular a parameter configuration, of the patient with corresponding patient information of a reference patient or a plurality of reference patients. A likelihood of a medical condition and/or an, in particular critical, change in the state of health of the patient may be derived in this way.

According to one aspect, the health information comprises:
a diagnosis relating to the state of health of the patient, and/or
a prognosis relating to the state of health of the patient, and/or
a recommendation for action relating to the state of health of the patient.

When taking historical and current patient information into account, the state of health of the patient may advantageously be determined for a current point in time, but may also be extrapolated into the future, e.g. in the context of a prognosis. Furthermore, recommended actions which may counteract an undesirable development in the state of health may be derived as a function of individual medical parameters.

In an exemplary embodiment, the first function comprises an algorithm, in particular an intelligent algorithm, and/or a model. The first function may be suitable in particular for processing, correlating and reformatting one or more medical parameters of the plurality of patient information and/or for evaluating the same in a comparison with patient information of a reference patient, a cohort of reference patients as well as arbitrary further information. The first function may be present for example in the form of a computer program and/or a data structure and be executed by means of the computing unit. The first function may further comprise a plurality of functions and/or algorithms which allocate and/or process individual tasks and/or operations of the first function.

A check is carried out as a function of the ascertained health information about the patient to determine whether a trigger condition has been fulfilled. The check may be conducted by means of the computing unit e.g. based on the first function and/or a further function. A trigger condition may be fulfilled for example in the event of an indication of a deterioration in the physical constitution of the patient, an unusual key indicator, a combination of key indicators and/or an atypical configuration of medical parameters of the patient. The trigger condition is in this case the deciding factor for a form in which the ascertained health information about the patient is provided.

In an exemplary embodiment, providing the ascertained health information about the patient comprises storing the health information about the patient in a memory unit of a computer, a notebook, a server and/or a cloud. However, it is equally conceivable that the health information about the patient is provided to a user, an output unit and/or a device, in particular a medical device and/or a private device of the patient. An output unit may be for example a screen, a monitor or a touchscreen which generates a visual output to a user. The private device of the patient may be in particular a smart device, such as e.g. a smartwatch, a smartphone or a tablet. A user, in this context, may be a physician or a treating medical practitioner who can refer to the ascertained health information, e.g. in order to derive a diagnosis, a possible treatment and/or a routine check. It is further conceivable that providing the ascertained health information about the patient comprises prioritizing the patient in a worklist of the user as a function of the ascertained health information about the patient. In particular, the output may be realized in the form of an alert message which draws the attention of the user to the health information about the patient. It is further conceivable that providing the health information comprises outputting a recommendation with regard to performing a medical test and/or a diagnostic method. By means of the recommended medical test and/or the diagnostic method it is possible in particular to obtain patient information which enables the state of health of the patient to be determined more effectively or more accurately. A medical test may constitute for example a blood test, a microbiological smear, a urine test or the like. In an exemplary embodiment, a diagnostic method comprises using an imaging method.

By providing the inventive method it is possible to realize an automatic detection of a change in the physical constitution of a patient. As a result, possible medical conditions of the patient may advantageously be detected already at a preliminary stage or at an early stage and appropriate measures for monitoring and/or preventive treatment initiated. Furthermore, early detection of medical conditions enables treatment outcomes to be improved and patient treatment costs to be reduced. Moreover, by applying the inventive method it is possible to provide medical monitoring of patients as a function of a plurality of patient information which is independent of a medical state of the patient. In this case, in particular medical parameters may be drawn upon which can be acquired with little effort during routine examinations. On this basis, a cost-effective, automated monitoring of the state of health of patients may advantageously be provided.

According to one aspect, the receiving of the plurality of patient information comprises at least a
receiving of first patient information from a first information source, and
receiving of second patient information from a second information source which is different from the first information source,
wherein the first information source and the second information source are selected from: a hospital information system, a radiological information system, a picture archiving and communication system, a laboratory information system, a pathology information system, a smart device of the patient, a diagnostic medical device, a patient registration, a patient health record, a recorded patient consultation, diagnostic findings, an input acquired by means of a user interface.

In an exemplary embodiment, the plurality of the patient information is assigned to two, at least three or at least four of the above-cited information sources. It is in particular conceivable that one information source of the at least two, at least three or at least four information sources is a smart device of a patient or diagnostic findings pertaining to the patient. In a further embodiment, the plurality of patient information is assigned to at least one diagnostic finding and one smart device of the patient. A diagnostic finding may in this case represent in particular a description and/or diagnosis of the patient produced by a medical practitioner. The diagnostic finding may be present e.g. as a file in an unstructured file format, such as e.g. a text document, an audio file or a video file.

The smart device of the patient may include for example a sensor for measuring medical parameters of the patient. It is equally conceivable that the smart device is coupled to a further device and/or to a corresponding sensor which transfers medical parameters of the patient to the smart device. For example, the smart device may comprise dedicated applications (apps) which analyze speech, gestures, a movement profile or the like of the patient by means of one or more suitable sensors. Patient information may be received from the smart device for example by means of the dedicated application via any wireless or wired connection. Accordingly, the plurality of patient information may be received from a database of a hospital information system, a radiological information system, a diagnostic medical device and/or a picture archiving and communication system. In an exemplary embodiment, such information sources are integrated into a communications network of a hospital, a practice or a clinical institution and enable a corresponding access to the plurality of patient information. A diagnostic medical device may for example comprise an imaging apparatus, a heart rate monitor, a molecular diagnostic device, an electrocardiogram monitor or the like.

Further information sources may include a patient registration, a patient health record or a recorded patient consultation, minutes of a meeting (e.g. a case review) as well as a description of symptoms of the patient. Such patient information may be linked to a database of the above-cited information sources or represent a separate information source. It is conceivable in particular that a part of the plurality of patient information, in particular the diagnostic findings, the recorded patient consultation and the patient registration, is present in an unstructured file format. Furthermore, the information source may also represent an input by a user, by a patient and/or by a relative of the patient by means of a user interface.

According to one aspect, the first patient information includes medical image data and the second patient information includes no medical image data, medical image data comprising in particular radiological image data and/or histopathological image data, and the second patient information comprising in particular laboratory data, vital signs data, an electronic patient health record and/or one or more clinical findings pertaining to the patient.

By using a plurality of patient information from multiple information sources, a greater cross-section of medical parameters of the patient can be considered in the monitoring of the state of health of the patient. As a result, the health information can advantageously be determined with a higher degree of accuracy and/or a higher level of reliability. Furthermore, by considering patient information of a smart device of the patient, an amount of effort expended for the acquisition of patient information can be reduced and a greater quantity of patient information can be provided.

According to one aspect, at least one piece of patient information from the received plurality of patient information comprises a pointer to an evolution over time of at least one medical parameter of the patient, further comprising the step of:
processing the plurality of patient information, comprising
   quantifying the evolution over time of the at least one medical parameter, and/or
   determining a normal value of the at least one medical parameter, a deviation of the at least one medical parameter from the normal value being determined in addition,
wherein the health information about the patient is ascertained as a function of the first function as well as of the evolution over time of the at least one medical parameter and/or of the deviation of the at least one medical parameter from the normal value.

A pointer to an evolution over time of a medical parameter of the patient may represent e.g. a continuous or discretely resolved progression over time of an arbitrary medical parameter, such as e.g. a blood pressure, a cardiac activity, but also a dimension and/or a volume of an organ or a pathological structure. The dimension and/or the volume of the organ or the pathological structure may be acquired for example by means of segmentation of image data of imaging methods at different points in time and the evolution thereof over time. Diagnostic findings for patients, on the other hand, typically contain a time stamp or a date which can be referred to in order to allow a quantification of the evolution over time of a symptom.

Quantifying the evolution over time of the at least one medical parameter may comprise e.g. determining a percentage and/or absolute change in a value of the at least one medical parameter in a predetermined period of time. However, it is equally conceivable that the quantification of the evolution over time of the at least one medical parameter comprises a comparison of the evolution of the at least one medical parameter with an evolution over time of a second medical parameter and/or a limit value. A limit value may in this case be a value specified by experts, the exceeding of which indicates a marked irregularity in the state of health of the patient. The at least one medical parameter may for example represent a measured value, such as e.g. a weight, a blood pressure and/or a urine value, a description of a symptom, but also a dimension and/or a volume of a physiological and/or pathological structure of the patient. For this purpose, the processing of the plurality of patient information may comprise a segmenting of images and/or image data of an imaging method. For example, a growth of a tumor may be quantified by segmenting the tumor in images of a number of magnetic resonance scans performed at different points in time.

A normal value may represent a mean value, a limit value or a statistically weighted average of a number of measured values of the at least one medical parameter. In particular, the normal value may be specified specifically for the patient based on the patient information. In an example, the normal value is defined by a limit below which 95% of the measured values of the at least one medical parameter of the patient lie. Exceeding this limit may therefore represent a marked change in the at least one medical parameter. For example, when a current set of the plurality of patient information is received, a deviation of a current measured value of the at least one medical parameter from the normal value may be determined. However, it is equally conceivable that a deviation of a measured value of the medical parameter is determined on the basis of a present time series of measured values. According to some embodiments, the normal value for a medical parameter may be dependent on further medical parameters. Thus, e.g. an age of a patient may necessitate different normal values. Information concerning a morbid tissue change, e.g. in the lung, may also produce an impact on a normal value for oxygen provision.

According to one aspect, the evolution over time of the at least one medical parameter of the patient is quantified and/or a normal value of the at least one medical parameter of the patient is determined as a function of a corresponding parameter of one or more reference patients. Accordingly, the method may comprise a step of determining one or more reference patients based on the patient information. Medical parameters which show a high deviation compared to corresponding parameters of a reference patient or a group of reference patients may in this case be characterized as distinctive parameters. Quantifying the evolution over time of the at least one parameter and/or determining the normal value of the at least one medical parameter of the patient may be carried out by means of the computing unit as a function of the first function.

By quantifying the evolution over time of the at least one parameter and/or determining the normal value of the at least one medical parameter of the patient, a reference base adapted to fit individual requirements of the patient may advantageously be provided. This also enables very slow changes in the state of health of the patient over an extended period of time to be identified in a robust manner By means of a comparison with corresponding parameters of reference patients, the at least one medical parameter may advantageously be determined as a function of a specific boundary condition, such as e.g. a population group, a nationality or a specific medical condition. As a result, influences which are significantly correlated with the specific boundary condition can advantageously be considered during the processing of the plurality of patient information.

According to one aspect, the method comprises the step of:
processing the plurality of patient information, wherein the processing of the plurality of patient information is carried out as a function of a sensor data fusion method.

The sensor data fusion method may be employed in particular in order to increase a quality, a reliability and/or a scale of the plurality of patient information. The sensor data fusion method may in this case comprise in particular a model and/or an algorithm which are configured to replace and/or correct missing and/or erroneous measured values of at least one parameter. It is furthermore conceivable that implausible measured values are supplemented and/or corrected by means of the sensor data fusion method as a function of other medical parameters of the patient. The sensor data fusion method may be further configured to correlate measured values of medical parameters of the patient in order to generate virtual parameters. Virtual parameters may for example register theoretical relationships between medical parameters or provide dependencies which are not directly accessible during a measurement on the patient. The sensor data fusion method may include classification methods, rule-based methods and/or stochastic methods in order to consolidate medical parameters with one another. It is conceivable in particular that the sensor data fusion method comprises using a Kalman filter, fuzzy logic and/or logical connections between medical parameters.

By using a sensor data fusion method, the quality, the completeness and/or the reliability of the plurality of patient information may advantageously be increased. Furthermore, by generating virtual parameters it is possible to avoid complex and time-consuming measurement methods for acquiring certain medical parameters, thereby enabling an enhanced quality of the plurality of patient information to be provided with little metrological overhead and/or at low cost.

According to one aspect, the method comprises the further step of:
processing (S2) the plurality of patient information (PI), wherein a part of the plurality of patient information (PI) is present in an unstructured file format and wherein the processing of the plurality of patient information (PI) comprises extracting the part of the plurality of patient information (PI) into a structured file format by means of the computing unit (SYS.CU), wherein the part of the plurality of patient information (PI) is extracted as a function of a computational linguistics method.

As described above, a part of the plurality of patient information may be present in an unstructured file format. Examples of patient information in unstructured file formats are diagnostic findings in text form, but also audio and/or video recordings of a patient consultation, a treatment and an examination. In an exemplary embodiment, patient information which is present in unstructured file formats is extracted and/or converted into structured file formats in order to allow a simple and reliable acquisition and processing of medical parameters. What is to be understood by the term structured file format in this context is any machine-readable file format which generally allows a structured storage and processing of data. Examples of such file formats are binary code, hexadecimal numbers, but also known high-level languages and specialized file formats, such as e.g. RDFa, HTML, CSV, XML, JSON, DICOM and the like, as well as file formats which implement the HL7 (Health Level 7) or FHIR (Fast Healthcare Interoperability Resources) standards.

It is conceivable that patient information available in unstructured file formats comprises a description of symptoms and/or medical parameters which are based on natural language. In an exemplary embodiment, the processing of natural language is accomplished by means of a computational linguistics method, such as e.g. by using a text mining method, a pipeline model and/or a semantic network, in particular an artificial neural network, a multilayer neural network (deep learning) or a MultiNet (multilayered extended semantic network). The processing of natural language may in this case include one or more of the following steps of a pipeline model:
speech recognition,
tokenization,
morphological analysis,
syntactic analysis,
semantic analysis, and/or
dialog analysis.

According to one aspect, patient information in an unstructured file format is processed by means of an artificial neural network or a multilayer neural network. It is furthermore conceivable that one or more of the listed steps of the pipeline model are performed by means of an artificial neural network or a multilayer neural network. Artificial neural networks and multilayer neural networks may advantageously be trained with the aid of large volumes of data in order also to process linguistically complex and/or colloquial formulations in a robust and reproducible manner In particular, artificial neural networks or multilayer networks may advantageously be trained to identify verbal ambiguities of the user, such as e.g. an incorrect naming or a paraphrasing of a medical parameter.

According to one aspect, patient information is processed as a function of a logical model and/or a statistic model. Such models may be integrated into the processing of patient information according to the pipeline model and perform and/or support individual steps or all of the above-listed steps. It is conceivable that patient information includes a predetermined selection of terms and/or keywords which can be recognized by means of statistical and/or logical models and assigned to a medical parameter.

Thanks to the possibility of processing unstructured file formats, a comprehensive set of patient information which normally is usable only by way of interpretation by a medical practitioner may advantageously be referred to automatically for monitoring the state of health of the patient and correlated with further medical parameters. In particular, this also enables patterns and/or correlations of medical parameters to be analyzed which usually remain unused during an examination of a patient.

According to one aspect, the method comprises the further step of:
processing the plurality of patient information, wherein the processing of the plurality of patient information comprises checking for the presence of at least one new piece of patient information and/or of an appointment, wherein the health information about the patient is ascertained and/or the health information about the patient is provided as a function of the presence of the at least one new piece of patient information and/or of the appointment.

It is conceivable that if at least one new piece of patient information is present, a trigger signal is provided, the health information about the patient being ascertained and/or the health information about the patient being provided automatically when the trigger signal is present. In an exemplary embodiment, the checking for the presence of at least one new piece of patient information is carried out during a processing of the plurality of patient information by means of the computing unit. An appointment may represent e.g. an appointment for a consultation with the patient, a time for a medical conference, a time for a case review and the like. In an exemplary embodiment, the presence of a corresponding appointment is checked by means of a query submitted to a medical information system, a reference to a diary of a physician or the like.

By limiting a performance of steps of the inventive method as a function of the presence of at least one new piece of patient information, it is advantageously possible to spare capacities of a communications and information infrastructure that is being used. Furthermore, an updated assessment of the state of health of the patient may advantageously be carried out so that a user is alerted immediately if a relevant development occurs.

According to one aspect, ascertaining the health information comprises:
correlating the determined parameter configuration with one or more reference parameter configurations, wherein the reference parameter configurations indicate health information in each case, and
ascertaining the health information based on the correlation step.

The parameter configuration of the patient may be determined, as described above, for example as a function of a normal value of a medical parameter being exceeded and/or as a function of a quantified evolution over time of the medical parameter. In this case, in particular further medical parameters which are related to the medical parameter may be considered as a parameter configuration. However, the further medical parameters may also reveal an exceeding of a normal value and/or a deviation from an expected or quantified evolution over time. The parameter configuration may therefore be characterized by a selection of interrelated medical parameters.

The health information may also be ascertained by means of a correlation of the determined parameter configuration with a reference parameter configuration. A reference parameter configuration may for example be assigned to one or more reference patients. The reference patients may in this case belong to a critical and/or a non-critical group, in particular with regard to a medical condition, a trend and/or an indication, and consequently indicate health information. This may mean that at least some of the reference patients have a confirmed medical condition (critical group) or are free from a medical condition (non-critical group). The medical condition of the critical group of reference patients may in this case be related to the determined parameter configuration.

According to one aspect, ascertaining the health information comprises:
correlating the determined parameter configuration with a comparison parameter
configuration of a reference patient, and
ascertaining the health information based on the correlation step.

It is equally conceivable that reference patients reveal a state of health which possesses no generally known relationship with the determined parameter configuration of the patient. In an exemplary embodiment, the correlation of the determined parameter configuration of the patient with a comparison parameter configuration of a reference patient is output to the user and/or the patient by means of an output unit.

By correlating a determined parameter configuration of the patient with a reference or comparison parameter configuration, a statistical relationship between the determined parameter configuration of the patient and a likelihood of a medical condition and/or an, in particular critical, change in the state of health of the patient may advantageously be derived. It is furthermore possible, by means of a suitable selection of the reference patients on the basis of specific boundary conditions, such as e.g. an age, a population group, a gender and/or a nationality, to determine the likelihood of the medical condition and/or the, in particular critical, change in the state of health of the patient as a function of boundary conditions which are adjusted to fit the patient.

According to one aspect, the method comprises the step of:
determining a priority level of the health information about the patient by means of the computing unit as a function of a second function as well as of the health information about the patient and/or the plurality of patient information.

In an exemplary embodiment, the priority level is determined by means of the computing unit as a function of the second function, the plurality of patient information and/or the health information about the patient. It is equally conceivable that the priority level is determined as a function of a distinctive medical parameter, a deviation of the distinctive medical parameter from the determined normal value and/or one or more corresponding parameters of reference patients. Determining the priority level of the health information about the patient may in particular comprise applying the second function to the health information about the patient. The second function may for example include algorithms which are configured to perform a rule-based determination of the priority level of the health information about the patient. It is conceivable that the second function determines the priority level of the health information as a function of an arbitrary trigger condition, a waiting time of the patient, an age of the patient, a state of health of the patient, as well as an arbitrary other piece of patient information and/or further pieces of patient information. In an exemplary embodiment, the second function is a second trained function. The second function or the second trained function may in this case constitute a part of the first function or an entity separate from the first function.

The priority level may represent a measure for a relevance of the ascertained health information about the patient. It is furthermore conceivable that the priority level represents a reliability and/or a degree of pertinence of the ascertained health information about the patient. In an example, a low priority level may be determined if a markedly increased medical parameter of the patient still lies in a normal range in comparison with a corresponding parameter of a plurality of reference patients. In a further example, a high priority level may be determined if a medical parameter of the patient deviates only marginally from the normal value of the patient but is increased in comparison with a critical group of reference patients. A dimension of a value of the priority level may in this case be dependent, inter alia, on the medical parameter in question, on a deviation of the medical parameter from a normal range, on an evolution over time of the medical parameter and/or on a relation of the parameter in question with a corresponding medical parameter of a reference patient.

According to one aspect, checking whether a trigger condition is fulfilled based on the ascertained health information about the patient is performed as a function of the determined priority level of the health information about the patient. This can mean that the priority level of the health information about the patient is referred to in order to check whether the trigger condition has been fulfilled. In an exemplary embodiment, the trigger condition is considered fulfilled if a high priority level is present. Analogously, the trigger condition may be regarded as not fulfilled if a low priority level is present. It is conceivable that the trigger condition is regarded as fulfilled if a predetermined limit value of the priority level is exceeded. The ascertained health information about the patient may in this case be output to the user. The second trained function may be interpreted as a decision-making entity concerning a fulfillment of the trigger condition. An output of the ascertained health information about the patient to the user and/or a notification of the user may therefore be accomplished as a function of individual boundary conditions of the patient which are quantifiable by means of the priority level. In an embodiment, the patient is prioritized in the worklist of the user as a function of the determined priority level of the health information about the patient.

By determining the priority level of the health information about the patient, the output to the user may advantageously be limited to cases in which an immediate examination and/or treatment of the patient is relevant. Furthermore, a reduction in the outputs of information about the state of health of the patient in non-critical cases enables capacities of the user, of a treating medical practitioner and/or of a diagnostic and communications infrastructure of a clinical institution to be spared.

According to one aspect, the second function is a second trained function, wherein providing the ascertained health information comprises outputting the ascertained health information about the patient to the user, further comprising the steps of:
acquiring an assessment of the user in respect of the priority level of the health information about the patient by means of the interface, and
adapting the second trained function by means of the computing unit at least as a function of the acquired assessment of the user in respect of the priority level of the health information about the patient and the ascertained health information about the patient.

In this embodiment, the priority level of the health information about the patient may also comprise, inter alia, an evaluation of an urgency, an importance and/or a relevance of the health information about the patient. As described above, the priority level may be determined by means of the computing unit as a function of the above-described second trained function as well as of the plurality of patient information and/or the health information about the patient. In an exemplary embodiment, the priority level is output to the user by means of an output unit when the ascertained health information about the patient is provided. The priority level of the health information about the patient may therefore provide a pointer to the user as to whether an immediate treatment and/or an immediate diagnostic examination of the patient are/is necessary.

According to one aspect, the output of the priority level of the health information about the patient comprises an invitation to the user to make an assessment of the priority level of the health information about the patient. Such an invitation may comprise an acoustic and/or visual output by means of a suitable output unit. In an example, the user is alerted by means of a warning signal to a presence of the ascertained health information about the patient and/or requested by means of a textual and/or symbolic message to evaluate and/or assess the priority level of the health information about the patient. It is conceivable that the user enters the assessment of the priority level of the health information about the patient via a suitable input unit, such as e.g. a mouse, a keyboard or a touchscreen. The input by the user may accordingly be acquired by means of the interface and used for adapting (e.g. training) the second trained function. The output of the health information about the patient to the user as well as the input by the user with the assessment of the priority level of the health information about the patient may in this case comprise any forms of communication. Conceivable among other things are an exchange of voicemails, text messages, a checking of checkboxes and/or an interaction with symbolic control elements (e.g. a “like” icon, a “thumbs-up” icon or the like).

The second trained function may be trained in particular by a user in order to adapt the priority level of the ascertained health information about the patient as a function of user requirements and/or requirements of a clinical institution. In one embodiment, the second trained function comprises a classification method, such as e.g. a nearest-neighbor classification. In an exemplary embodiment, in this case, the second trained function is adapted by means of the computing unit as a function of the acquired assessment of the user in respect of the priority level of the health information about the patient and the ascertained health information about the patient. In a further embodiment, the second trained function comprises an artificial neural network or a multilayer neural network. In an exemplary embodiment, the second trained function is adapted by means of the computing unit as a function of the acquired assessment of the user in respect of the priority level of the health information about the patient, the ascertained health information about the patient and the determined priority level of the health information about the patient.

By means of an output of the priority level of the health information about the patient, a user may be alerted to a necessary observation of the state of health of a patient. This advantageously enables a risk of an incorrect assessment of the state of health of the patient, e.g. due to a slow progressive change in medical parameters of the patient or a configuration of medical parameters without a specific medical indication, to be avoided.

According to one aspect, providing the ascertained health information about the patient comprises outputting the ascertained health information about the patient to the user, wherein the first function is a first trained function, further comprising the steps of:
registering feedback of the user in respect of a validity of the health information about the patient by means of the interface, and
adapting the first trained function by means of the computing unit at least as a function of the plurality of patient information of the patient as well as of the registered feedback of the user in respect of the validity of the health information about the patient.

Feedback of the user in respect of the validity of the health information about the patient may for example comprise an evaluation, an assessment, a correction and/or arbitrary feedback on the health information about the patient. By means of the feedback in respect of the validity of the health information about the patient, the user may be able to correct an incorrect assessment and/or an inaccuracy of at least a part of the ascertained health information about the patient. However, it is equally conceivable that the first trained function, the second trained function and/or a third trained function are/is configured to determine a correction of the ascertained health information about the patient by means of the computing unit in accordance with the evaluation, the assessment or the feedback of the user on the health information about the patient. The feedback of the user in respect of the validity of the health information about the patient may be registered, as described above, by means of a suitable input unit.

According to one aspect, the first trained function is adapted by means of the computing unit as a function of the plurality of patient information of the patient as well as of the registered feedback of the user in respect of the validity of the health information about the patient. The first trained function may in this case comprise in particular a classification method, such as e.g. a nearest-neighbor classification. In a further embodiment, the first trained function comprises an artificial neural network or a multilayer neural network. In such neural networks, the first trained function may be adapted by means of the computing unit in particular as a function of the plurality of patient information, the ascertained health information and the acquired validity of the health information.

By registering the feedback of the user in respect of the validity of the health information about the patient, the first trained function may advantageously be adapted to match a knowledge level of the user and/or errors or inaccuracies of the first trained function may be corrected. This enables an accuracy of the ascertained health information about the patient to be continuously improved and/or adapted in line with current medical knowledge.

According to one aspect, the first function is a first trained function and/or the second function is a second trained function, the first trained function and/or the second trained function being based on an artificial neural network, a multilayer neural network, a convolutional neural network, a nearest-neighbor classification, a support vector machine and/or a Bayesian network.

The first trained function and/or the second trained function may in particular represent separate or interrelated functions or modules of an evaluation algorithm. In an exemplary embodiment, the first trained function and the second trained function are different from one another. The first trained function and the second trained function may in particular comprise different functions of the above-cited functions. In an example, the first trained function is configured to perform a nearest-neighbor classification in order to determine the health information about the patient. In this case a first part of the plurality of patient information may be sourced from a database for which health information about the patient is already present. For a second part of the plurality of patient information, in contrast, the health information about the patient is determined by means of the first trained function as a function of the first part of the plurality of patient information from the database. In this case, distance metrics, such as e.g. a Euclidian distance, a Manhattan metric or the like, may be used in order to determine a distance between a medical parameter of the second part of the plurality of patient information and corresponding medical parameters of the first part of the plurality of patient information. The medical parameter of the second plurality of patient information may subsequently be assigned health information using a variable k which defines a greatest number of neighbors.

According to one aspect, the first trained function comprises an artificial neural network having a convolution layer and/or a deconvolution layer. The artificial neural network may additionally comprise a pooling layer. In particular, the artificial neural network may be a convolutional neural network or a multilayer convolutional neural network (deep convolutional neural network). The artificial neural network may be configured in particular to determine health information about the patient as a function of diagnostic images, such as e.g. a magnetic resonance tomography image, an X-ray image, a computed tomography image and/or corresponding image datasets of imaging devices. It is furthermore conceivable that the first trained function comprises identifying and/or segmenting physiological and/or pathological structures of the patient as a function of the diagnostic images. The first trained function may be further configured to perform a survey of the pathological and/or physiological structures in order to determine a volume and/or a dimension of the pathological and/or physiological structure.

According to one aspect, the first trained function comprises an artificial neural network or a multilayer neural network. The multilayer neural network may in this case have a number of hidden layers, e.g. two layers, three layers, four layers or more than four layers. It is conceivable that the artificial neural network or multilayer neural network is trained to ascertain the health information about the patient as a function of the plurality of patient information. The first trained function may additionally be configured to correct or update the health information about the patient as a function of the feedback of the user in respect of the validity of the ascertained health information about the patient.

According to one aspect, the second trained function comprises an artificial neural network or a multilayer neural network which is trained to determine the priority level of the health information about the patient as a function of the health information about the patient and/or the plurality of patient information. It is equally conceivable that the artificial neural network is trained to correct or update the priority level of the health information about the patient by means of the computing unit as a function of the assessment of the user in respect of the priority level of the health information about the patient.

The first trained function and/or the second trained function may of course include further intelligent algorithms and/or classification methods, such as e.g. a support vector machine, or an expert system, such as e.g. a Bayesian network. A Bayesian network may be configured for example to ascertain the health information about the patient as a function of a probability model.

By using intelligent algorithms, such as e.g. neural networks, expert systems and/or learning classification methods, the user is able to influence a workflow and/or a result of the inventive method, e.g. by means of the assessment of the priority level and/or the feedback in respect of the validity of the health information about the patient. Accordingly, new medical knowledge, but also errors or inaccuracies of the method, can advantageously be considered in the monitoring of the state of health of the patient. Furthermore, a trained function can adapt in a self-learning manner to the available patient information. To that extent, the trained function fulfills the task of an intelligent filter which, from the set of available information, adaptively and patient-specifically extracts and correlates the parameters and variables that are relevant to the derivation of health information.

A method for adapting a first trained function comprises the following steps of:
receiving the first trained function by means of an interface,
receiving a plurality of patient information of a patient and first information about a state of health of a patient by means of the interface, wherein the plurality of patient information includes at least two different medical parameters assigned to the patient, adapting the first trained function by means of the computing unit as a function of the plurality of patient information of the patient and the first health information about the patient.

The first trained function may be received by means of an interface of a training system. The first trained function may in this case be transferred to the interface of the training system by means of a wireless or wired connection. The interface may be configured in particular as a communications interface.

The interface may also receive training data, such as e.g. the plurality of patient information of the patient and the first health information about the patient. The first health information about the patient may in this case be based on a diagnosis, on findings or on an assessment of a medical practitioner, but also on a verified result of a medical examination or guidelines of an expert community. In an exemplary embodiment, the first health information about the patient is assigned to the training data of the plurality of patient information and/or correlated therewith. This may mean that a point in time of a generation of the first health information about the patient substantially coincides with a point in time of an acquisition of the plurality of patient information of the patient.

The first trained function is adapted by means of the computing unit of the training system as a function of the plurality of patient information of the patient and the first health information about the patient. In an embodiment, the first trained function comprises a nearest-neighbor classification. The adapting of the first trained function may in this case comprise in particular a storing of the training data (lazy learning) or a normalizing of the training data.

By providing a first trained function which comprises a classification method, a particularly simple and efficient method for ascertaining the health information about the patient can be provided. Corresponding classification methods are associated in particular with a low computational overhead and may also be reliably operated with an older information technology and/or communications technology infrastructure.

A further aspect relates to a method for adapting the second trained function, comprising the following steps of:
receiving the second trained function by means of an interface,
receiving first information about a state of health of a patient,
determining a priority level of the first health information about the patient by means of the computing unit as a function of the second trained function,
acquiring an assessment in respect of the determined priority level of the first health information about the patient by means of the interface, and
adapting the second trained function by means of the computing unit as a function of a comparison of the priority level of the first health information about the patient and the assessment in respect of the determined priority level of the first health information about the patient.

The assessment in respect of the determined priority level of the first health information about the patient may in this case comprise a predetermined assessment of a training dataset and/or an assessment of a user.

By providing a second trained function, a particularly simple and efficient adjustment of the priority level of the ascertained health information about the patient can be provided as a function of an input by the user.

According to one aspect, the method for adapting the first trained function additionally comprises the step of:
ascertaining second health information about the patient by means of the computing unit as a function of the plurality of patient information and the first trained function,
wherein the first trained function is further adapted by means of the computing unit as a function of the second health information about the patient.

The first trained function and the training data may be received, as described above, by means of the interface of the training system. The second health information about the patient may subsequently be ascertained by means of the computing unit of the training system as a function of the training data and the first trained function.

In an exemplary embodiment, the first trained function is adapted as a function of the first health information about the patient, the second health information about the patient and the plurality of patient information. In an embodiment, the first trained function comprises an artificial neural network or a multilayer neural network. Such neural networks may be trained in particular on the basis of a comparison of a target output (e.g. first health information about the patient) and an actual output (e.g. second health information about the patient) within the scope of a supervised learning process. For this purpose, a change to be performed to a configuration of the neural network may be inferred as a function of mathematical methods, such as e.g. a delta rule, a backpropagation method or an SGD (stochastic gradient descent) method. Changes to the configuration of the artificial neural network may in this case comprise:
developing new connections between neurons,
adjusting a weighting of the neurons,
adjusting a threshold value of the neurons,
adding or deleting neurons and/or connections between neurons, and
modifying an activation, a propagation and/or an output function.

The cited terms are known to the person skilled in the art and shall not be explained further here. Other learning methods are of course conceivable in addition to supervised learning, such as e.g. unsupervised learning or reinforcement learning.

The training system may be configured in particular to adapt a trained function according to a previously described embodiment of a method according to the disclosure. The training system is further configured to implement said methods and their aspects in that the interface and the computing unit are configured to perform the corresponding method steps.

By training a trained function on the basis of training data, the inventive method for providing the health information about the patient can be adapted in an efficient manner to fit specific requirements of a user and/or in line with new medical knowledge. Furthermore, the inventive method may advantageously contribute toward less experienced users learning and/or benefiting from adaptations based on training data of experienced users, experts and/or expert communities.

One aspect relates to a training system for adapting a first trained function, comprising:
an interface, configured to receive the first trained function, further configured to receive a plurality of patient information and first information about a state of health of a patient, the plurality of patient information including at least two different medical parameters assigned to the patient, and
a computing unit, configured to ascertain second health information about the patient as a function of the plurality of patient information and the first trained function, further configured to adapt the first trained function based on a comparison of the first health information about the patient and the second health information about the patient.

The first health information may in this case be correlated in particular with the plurality of patient information.

A further aspect relates to a training system for adapting a second trained function, comprising:
an interface which is configured to receive the second trained function and first health information about the patient,
a computing unit which is configured to determine a priority level of the first health information about the patient as a function of the second trained function and the first health information about the patient,
wherein the interface is further configured to acquire an assessment in respect of the determined priority level of the first health information about the patient, and
wherein the computing unit is further configured to adapt the second trained function as a function of a comparison of the priority level of the first health information about the patient and the assessment in respect of the determined priority level of the first health information about the patient.

The assessment in respect of the determined priority level of the first health information about the patient may in this case comprise a predetermined assessment of a training dataset and/or an assessment of a user.

According to one aspect, the interface of the training system according to the disclosure may be further configured to receive an assessment of a priority level of the first health information about the patient determined on the basis of the second trained function and/or feedback in respect of a validity of the ascertained health information about the patient determined on the basis of the first trained function, the second trained function and/or a third trained function. In an exemplary embodiment, the training system is configured to adapt the first trained function and the second trained function according to the inventive training system for adapting the first trained function and the inventive training system for adapting the second trained function.

Such a training system may be configured in particular to perform an inventive method for providing information about a state of health of a patient and/or an inventive method for adapting a trained function as well as their aspects. In an exemplary embodiment, the training system is configured to perform these methods and their aspects in that the interface and the computing unit are configured to perform the corresponding method steps.

The system comprises a computing unit, a determination unit and a user interface, wherein the determination unit has an interface and a first function, wherein the interface is configured to receive a plurality of patient information, and wherein the first function is configured to ascertain information about a state of health of a patient by means of the computing unit as a function of the plurality of patient information, wherein the computing unit is further configured to coordinate and perform an inventive method according to an above-described embodiment and to provide the ascertained health information about the patient by means of the user interface.

The system may in particular comprise a local computer, a notebook, a network computer, a server, a cloud, a tablet, a smart device or a comparable component or be connected to such a device. In an exemplary embodiment, a user, such as e.g. a medical practitioner or a member of a medical team, may access the system locally and/or by means of a remote connection in order to initiate a method according to the disclosure and/or to receive ascertained information about a state of health of a patient by means of the user interface. It is equally conceivable that the user may input an assessment of a priority level and/or feedback in respect of a validity of the ascertained health information about the patient locally or by means of a suitable input unit of the user interface.

In an exemplary embodiment, the interface of the determination unit is configured as a communications interface which is configured to receive or retrieve the plurality of patient information from one or more information sources. The plurality of patient information may be transferred by means of the interface to the first function and/or to a further function, such as e.g. a second trained function and/or a third trained function. In an exemplary embodiment, the computing unit is configured to coordinate communications links between the interface, the user interface and/or the determination unit. The computing unit is further configured to ascertain the health information about the patient by means of the first function as a function of the plurality of patient information. However, it is equally conceivable that the system has a plurality of computing units. For example, a first computing unit may be configured to ascertain the health information about the patient by means of the first function. A second computing unit and/or a further computing unit may be configured to coordinate a data exchange between the interface, the user interface, the determination unit and/or the first computing unit.

According to one aspect, the first function is a first trained function which is configured to determine the health information about the patient by means of the computing unit according to one of the above-described embodiments of the inventive method for providing the health information about the patient. In a further embodiment, the inventive system further includes a second trained function which is configured to determine the priority level of the health information about the patient by means of the computing unit according to one of the above-described embodiments of the inventive method for providing the health information about the patient. It is conceivable in particular that the first trained function and/or the second trained function may be adapted by the user according to one of the above-described embodiments of the inventive method for providing the health information about the patient. In an exemplary embodiment, the first trained function and/or the second trained function are based on an artificial neural network, a multilayer neural network, a convolutional neural network, a nearest-neighbor classification, a support vector machine and/or a Bayesian network.

In addition to the computing unit, the interface and the user interface, the system according to the disclosure may comprise further components for acquiring, processing and storing data, such as e.g. the plurality of patient information, a medical parameter, the health information about the patient, diagnostic images, inputs of the user and the like. For example, the system may comprise a controller, a main memory and a memory unit. The computing unit and/or the controller may for example comprise a microcontroller, a CPU, a GPU or the like. The main memory and/or the memory unit may include memory technologies, such as e.g. RAM, ROM, PROM, EPROM, EEPROM, flash memory, but also HDD storage, SSD storage or the like. It is conceivable that the memory unit constitutes an internal database which is electrically and/or mechanically connected to the computing unit of the system. However, it is equally conceivable that the memory unit is an external database which is connected to the computing unit by means of a network connection. Examples of external memory units are network servers with corresponding data storage facilities, as well as a memory unit of a cloud. The data may be transferred between the components of the system by means of analog and/or digital signals as well as suitable wired and/or wireless signal connections. For acquiring and processing voicemails and/or other inputs by the user, the system and/or the user interface may in particular comprise a voice input unit, a speech processing unit and/or an output unit as further components.

The components of the inventive system may advantageously be coordinated with one another, thereby enabling an inventive method for providing the health information about the patient to be performed in a time-efficient and robust manner In particular, the inventive system may be configured to coordinate and perform an execution of individual method steps autonomously. The health information about the patient may therefore be advantageously ascertained automatically and/or without specialized technical knowledge on the part of the user.

The computer program product can be loaded directly into a memory unit of an inventive system and/or an inventive training system and has program sections for performing all the steps of a method for providing information about a state of health of a patient according to an above-described embodiment and/or of a method for adapting a first trained function according to an above-described embodiment when the program sections are executed by the system and/or the training system.

The computer program product enables a method according to the disclosure to be performed quickly and in an identically reproducible and robust manner The computer program product is configured in such a way that it is able to perform the inventive method steps by means of a computing unit. The computing unit must in this case fulfill the respective requirements, such as, for example, having a suitable random access memory, a suitable graphics card or a suitable logic unit, so that the respective method steps can be carried out efficiently. The computer program product is stored for example on a computer-readable medium or held resident on a network, a server or a cloud, from where it can be downloaded into a processor of a local computing unit. Control information of the computer program product may also be stored on an electronically readable data medium. The control information of the electronically readable data medium may be configured in such a way that it performs an inventive method when the data medium is used in the computing unit of the imaging device. Examples of electronically readable data media are a DVD, a magnetic tape, a USB stick or any other data storage media on which electronically readable control information, in particular software, is stored. When said control information is read from the data medium and transferred to a controller and/or to the computing unit of the system, all the inventive embodiments of the above-described inventive methods may be implemented.

The computer-readable storage medium on which program sections that can be read and executed by a system and/or a training system are stored in order to perform all the steps of a method for providing information about a state of health of a patient according to an above-described embodiment and/or all the steps of a method for adapting a first trained function according to an above-described embodiment when the program sections are executed by the system and/or the training system.

An implementation realized to a large extent in the form of software has the advantage that systems and/or training systems already used previously in the prior art can also be easily upgraded by means of a software update in order to operate in the manner according to the disclosure. In addition to the computer program, such a computer program product may where necessary comprise additional constituent parts such as e.g. a set of documentation and/or additional components, as well as hardware components, such as e.g. hardware keys (dongles, etc.) to enable use of the software.

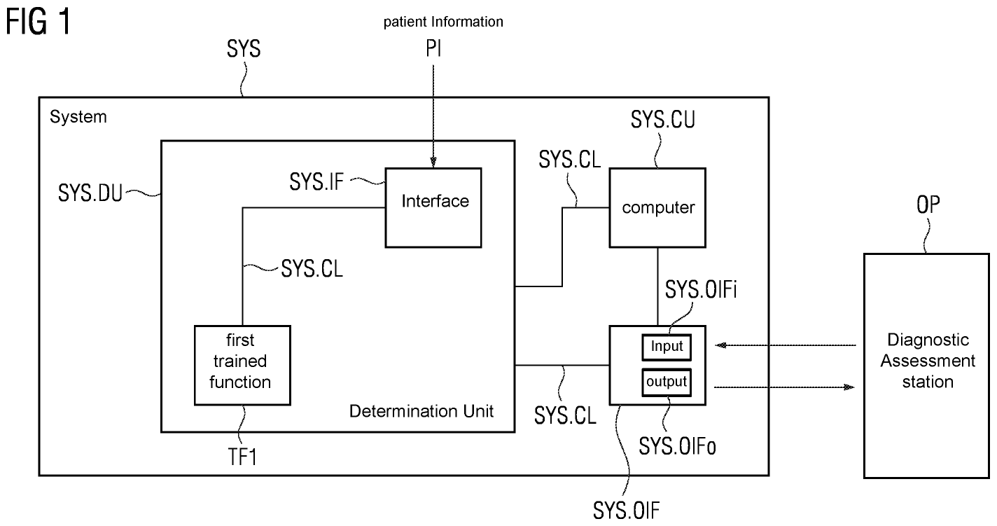

FIG. 1 shows a schematic view of a medical information network which comprises a system SYS for monitoring a state of health of one or more patients as well as at least one front-end computing device OP, between which a communications link exists. In an exemplary embodiment, the system SYS (and/or one or more components therein) includes processing circuitry configured to perform one or more functions and/or operations of the system SYS. Additionally, the system SYS may include one or more internal and/or external memories configured to store data, such as control data, computer code executable processing circuitry, patient data or information, image data, or other data or information as would be understood by one of ordinary skill in the arts.

The front-end computing device OP is configured as a diagnostic assessment station OP or diagnostic assessment workstation at which a user may view and analyze patient information PI as well as produce, check, amend and review medical findings. For this purpose, the diagnostic assessment station OP may have a user interface (not shown). The diagnostic assessment station OP may include a processor. The processor may comprise a central processing unit (CPU), a graphics processing unit (GPU), a digital signal processor (DSP), an image processing processor, an integrated (digital or analog) circuit or combinations of the aforementioned components and further devices for hosting a worklist, displaying patient information PI and supporting a user interaction. The diagnostic assessment station OP may for example comprise a desktop PC, a laptop or a tablet. In an exemplary embodiment, the front-end computing device OP (and/or one or more components therein) includes processing circuitry configured to perform one or more functions and/or operations of the front-end computing device OP. Additionally, the front-end computing device OP may include one or more internal and/or external memories configured to store data, information, and/or code as would be understood by one of ordinary skill in the arts.

According to one embodiment, the system SYS is configured for monitoring the state of health of one or more patients. The system SYS may be implemented as a single component or comprise a group of computers, like, say, a cluster. The system SYS may be configured as a cloud server. The system SYS may in particular comprise a real or virtual group of computers and/or storage devices. Depending on embodiment, the system SYS may be realized as a local server or as a cloud server. It is additionally conceivable that the system SYS is implemented on a notebook, a stationary computer and/or an operator control terminal of a medical institution. The system SYS may receive a plurality of patient information PI from different information sources, such as e.g. a hospital information system, a radiological information system, a picture archiving and communication system, a diagnostic medical device, a digital patient health record, a smart device of the patient and/or another information source, by means of the interface SYS.IF of the determination unit SYS.DU.

The plurality of patient information PI may be processed by means of the first function F1. The first function may be in particular a first trained function TF1. The first function F1 is configured to ascertain health information about the patient by means of the computing unit (computing device) SYS.CU as a function of the plurality of patient information PI. The computing unit SYS.CU may in this case represent a computing unit assigned to the system SYS or a dedicated computing unit of the determination unit SYS.DU (not shown). It is equally conceivable that the system SYS comprises a plurality of computing units, wherein at least one computing unit SYS.CU is configured to ascertain the health information about the patient on the basis of the first function F1. The computing unit SYS.CU and/or a further computing unit of the system SYS may be further configured to perform an inventive method for providing the health information about the patient. It is furthermore conceivable that the computing unit SYS.CU and/or the further computing unit of the system SYS coordinate a communication and/or a data exchange between the interface SYS.IF, the user interface SYS.OIF and/or the determination unit SYS.DU.

In the present embodiment, the system SYS comprises a user interface SYS.OIF having an output unit SYS.OIFo which comprises for example a screen, a monitor or a touchscreen. In an exemplary embodiment, the health information about the patient may be transferred to the output unit SYS.OIFo in order to generate a corresponding output to a user of the system SYS. The user may thus be kept informed about the state of health of the patient. It is equally conceivable that the user interface SYS.OIF has an input unit SYS.OIFi, such as e.g. a keyboard, a mouse, a microphone or the like in order to receive an input of the user. In one embodiment, the user may manually initiate a performance of the inventive method for providing the health information about the patient by means of the system SYS. For this purpose, the user may execute an inventive computer program product on the system SYS. In an exemplary embodiment, the system SYS provides a graphical user interface by means of the output unit SYS.OIFo, which graphical user interface can be controlled and/or parameterized by the user by means of the input unit SYS.OIFi. However, it is equally conceivable that the inventive method for providing the health information about the patient is performed automatically by means of the system SYS, e.g. when a new set of patient information and/or a new medical parameter are received, but also as a function of a predetermined criterion. The system SYS may be configured in particular to notify the user by means of the output unit SYS.OIFo when new health information about the patient has been ascertained.

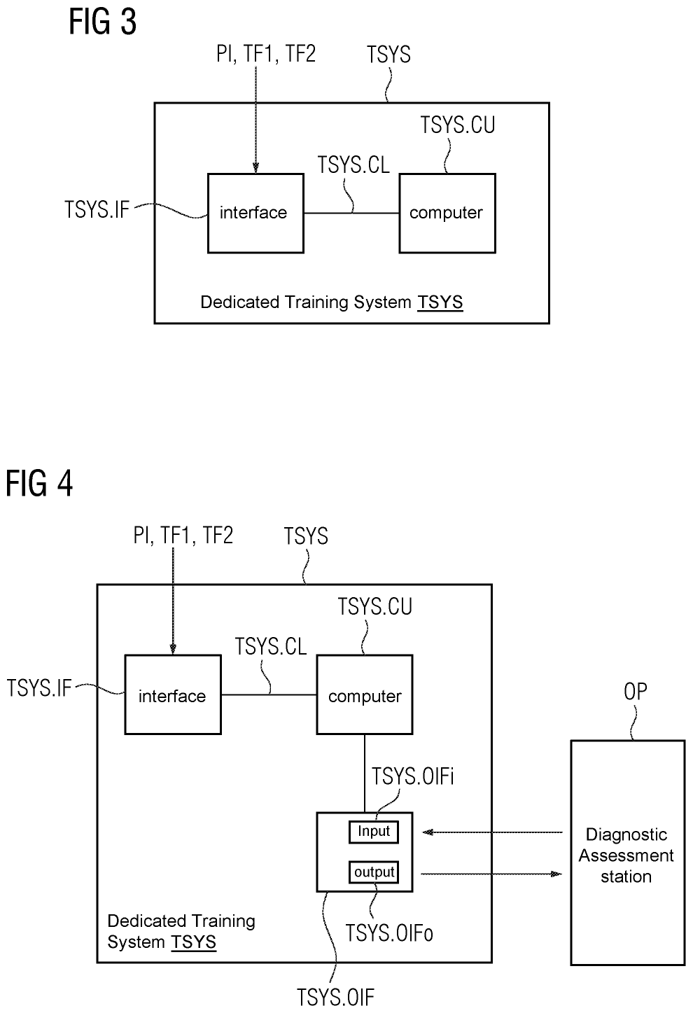

In an exemplary embodiment, the first function F1 is a first trained function TF1 which may be adapted e.g. by means of an inventive method for adapting the first trained function TF1. The first trained function TF1 may in particular be trained by means of a dedicated training system TSYS (see FIG. 3, FIG. 4) and/or an input of the user by means of the input unit SYS.OIFi. In one example, the input unit SYS.OIFi is configured to receive feedback of the user in respect of a validity of the health information about the patient. The first trained function TF1 may therefore by adapted by means of the computing unit SYS.CU as a function of the feedback of the user in respect of a validity of the health information about the patient according to an above-described embodiment of an inventive method for providing the health information about the patient and/or of a method for adapting a trained function TF1 and/or TF2.

The schematic view of the system SYS shown in FIG. 1 is to be understood as serving by way of example. A layout or topology of the system SYS may differ from the exemplary illustration shown in FIG. 1 without leaving the scope of protection of the disclosure. For example, the interface SYS.IF and the user interface SYS.OIF may be integrated in a common interface SYS.IF. It is equally conceivable that the system SYS includes a controller which controls a workflow and/or a data exchange between components of the system SYS, but also of the method for monitoring the state of health of the patient and/or of the method for adapting a trained function TF1 and/or TF2. In a further example, the computing unit SYS.CU may also be assigned to the determination unit SYS.DU. In the illustrated embodiment, the system SYS furthermore has a plurality of communication links SYS.CL which enable a communication between the components of the system SYS. The communication between the components may in this case be realized on a wireless or wired basis. In an exemplary embodiment, the components of the system SYS are electrically connected to one another by means of communication links SYS.CL. In an exemplary embodiment, the determination unit SYS.DU (determiner) may include processing circuitry that is configured to perform the function and/or operations of the determination unit.

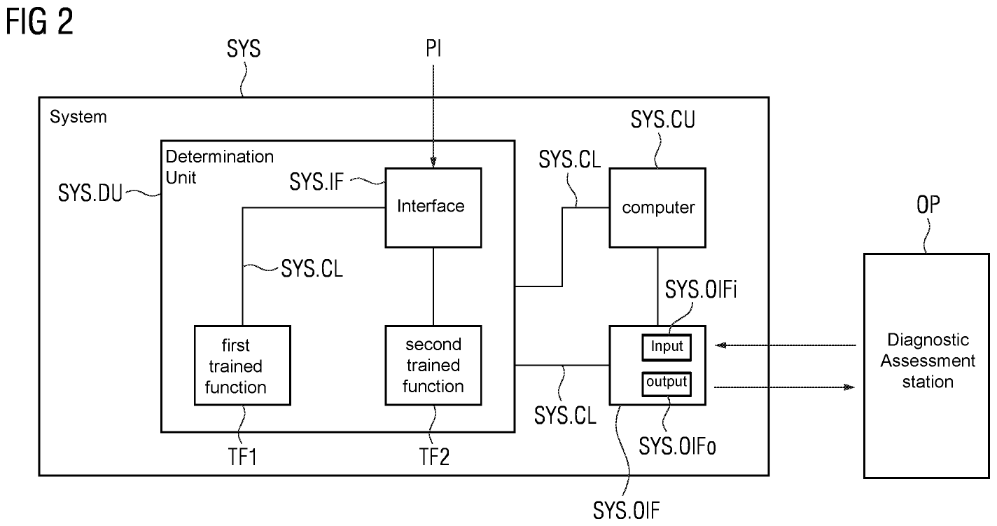

FIG. 2 shows a schematic view of a further embodiment of the inventive system SYS. In this example, the determination unit SYS.DU has a second function F2 which is configured as a second trained function TF2. In the present case, the second trained function TF2 is configured to determine a priority level of the health information about the patient by means of the computing unit SYS.CU. The priority level of the health information about the patient may be transmitted to the output unit SYS.OIFo of the user interface SYS.OIF for example by means of the determination unit SYS.DU or the computing unit SYS.CU and output to the user. It is conceivable in particular that the input unit SYS.OIFi of the user interface SYS.OIF is configured to acquire an assessment of the user in respect of the priority level of the health information about the patient and to forward the same to the determination unit SYS.DU. In an exemplary embodiment, the system SYS is configured to adapt the second trained function TF2 by means of the computing unit SYS.CU according to an above-described embodiment of the method for monitoring the state of health of the patient and/or of a method for adapting the second trained function TF2. However, the first trained function TF1 and/or the second trained function TF2 may, of course, also be configured as first function F1 and/or second function F2.

FIG. 3 shows a schematic view of an inventive training system TSYS according to an exemplary embodiment. In the present example, the training system TSYS comprises an interface TSYS.IF, which may be configured as a communications interface. The interface TSYS.IF is configured to receive a first trained function TF1, first health information about the patient, as well as a plurality of patient information PI from different information sources. The training system TSYS further comprises a computing unit TSYS.CU which is configured to ascertain second health information about the patient as a function of the plurality of patient information PI and the first trained function TF1. The computing unit TSYS.CU is furthermore configured to adapt the first trained function TF1 based on a comparison of the first health information about the patient and the second health information about the patient. In an exemplary embodiment, the first trained function TF1 comprises an artificial neural network, a multilayer neural network and/or a convolutional neural network which are adapted by the training system TSYS by means of supervised learning, unsupervised learning or reinforcement learning. In an exemplary embodiment, the training system TSYS (and/or one or more components therein) includes processing circuitry configured to perform one or more functions and/or operations of the training system TSYS. Additionally, the training system TSYS may include one or more internal and/or external memories configured to store data, information, and/or code as would be understood by one of ordinary skill in the arts.

In one example, the adapting comprises a supervised learning process. In this case, training data, such as e.g. the plurality of patient information PI, as well as a desired output, such as e.g. the first health information about the patient, are forwarded together with the first trained function TF1 to the training system TSYS. Based on a comparison of a target output (first health information about the patient) and an actual output (second health information about the patient) of the first trained function TF1, a change to a configuration of the first trained function TF1 may be inferred as a function of mathematical methods, such as e.g. a delta rule, a backpropagation method or an SGD method.

With the backpropagation method, for example, a difference is formed between the actual output and the target output of the multilayer neural network, which difference is considered an error. The error may subsequently be propagated back from an output layer to an input layer of the multilayer neural network. In this case a configuration of the multilayer neural network, in particular a weighting of connections between neurons, may be changed as a function of its effect on the error. Corresponding methods may be employed to minimize the error between the target output and the actual output of the multilayer neural network for an input pattern. Following the adaptation, the adapted first trained function TF1 may be output by means of the interface TSYS.IF.

In one embodiment, the interface TSYS.IF of the training system TSYS is further configured to receive a second trained function TF2. The computing unit TSYS.CU is in this case configured to determine a priority level of the first health information about the patient as a function of the second trained function TF2 and the first health information about the patient. In an exemplary embodiment, training data is also received by means of the interface TSYS.IF for the purpose of adapting the second trained function TF2. Such training data may comprise an assessment of the determined priority level of the first health information about the patient. In one example, the assessment of the determined priority level of the first health information about the patient includes a pattern having a priority level of the first health information about the patient that is deemed correct. The computing unit TSYS.CU of the training system TSYS is configured to adapt the second trained function TF2 as a function of a comparison of the priority level of the first health information about the patient and the assessment in respect of the determined priority level of the first health information about the patient.

In one example, the second trained function TF2 may comprise an artificial neural network, a multilayer neural and/or a convolutional neural network, which are trained by the training system TSYS by means of supervised learning, unsupervised learning or reinforcement learning. For example, the second trained function TF2 comprises a multilayer neural network which is adapted by means of a backpropagation method. In this case, a difference is formed between the actual output (priority level of the first health information about the patient) and the target output (assessment of the determined priority level of the first health information about the patient) of the multilayer neural network, which difference is considered an error. The error may subsequently be propagated back from an output layer to an input layer of the multilayer neural network. In this case a configuration of the multilayer neural network, in particular a weighting of connections between neurons, may be changed as a function of its effect on the error. Corresponding methods may be employed to minimize the error between the target output and the actual output of the multilayer neural network for an input pattern. After being adapted, the adapted second trained function TF2 may be output by means of the interface TSYS.IF.

The training system TSYS shown in FIG. 3 may, or course, also be a dedicated training system TSYS which is configured exclusively for adapting the first trained function TF1 or the second trained function TF2. It is further conceivable that the first trained function TF1 and/or the second trained function TF2 comprise a classification method, such as e.g. a nearest-neighbor classification or a support vector machine, and/or an expert system, such as e.g. a Bayesian network. In a simple example, the first trained function TF1 comprises a nearest-neighbor classification. Instead of ascertaining the second health information about the patient, the computing unit TSYS.CU may be correspondingly configured to store training data, such as e.g. the plurality of patient information PI and the first health information about the patient, but also to normalize training data.

FIG. 4 shows a schematic view of the training system TSYS, which includes a user interface TSYS.OIF. The user interface TSYS.OIF may comprise an output unit TSYS.OIFo and an input unit TSYS.OIFi. In an exemplary embodiment, the output unit TSYS.OIFo is configured to output the priority level of the health information about the patient determined by means of the second trained function TF2 to a user. The latter may input an assessment of the priority level of the health information about the patient by means of the input unit TSYS.OIFi in order to adapt the second trained function TF2. The second trained function TF2 may be adapted as described above. It is equally conceivable that the first trained function TF1 is adapted in that the user inputs feedback in respect of the validity of the second health information about the patient by means of the input unit TSYS.OIFi. The output unit TSYS.OIFo may be configured in particular to output an invitation for an input of the assessment of the priority level of the health information about the patient and/or the feedback in respect of the validity of the second health information about the patient to the user.

FIG. 5 shows a flowchart of a method for adapting the first trained function TF1.

In step T1, the first trained function TF1 is received by means of the interface TSYS.IF. In this case the first trained function TF1 may be received for example by a system SYS or a determination unit SYS.DU of a system SYS.

In step T2, the plurality of patient information PI of the patient and the first health information about the patient are received by means of the interface TSYS.IF, the plurality of patient information PI comprising a pointer to an evolution over time of at least one medical parameter of the patient. The plurality of patient information PI and the first health information about the patient may in this case represent a training dataset which can be read in from an internal data storage medium of the training system TSYS and/or from an external data storage medium.

In an optional step T3, the second health information about the patient is ascertained by means of the computing unit TSYS.CU as a function of the plurality of patient information PI and of the first trained function TF1. This optional step is performed in particular when the first trained function TF1 comprises an artificial neural network, a multilayer neural network and/or a convolutional neural network.

In step T4, the first trained function TF1 is adapted by means of the computing unit TSYS.CU at least as a function of the plurality of patient information PI of the patient and of the first health information about the patient. The first trained function TF1 may in this case comprise in particular a classification method, such as e.g. a nearest-neighbor classification or a support vector machine. In an exemplary embodiment, when the nearest-neighbor classification is used, the training data is stored and/or normalized. The first trained function TF1 may subsequently be output together with the stored and/or normalized training dataset.

It is further conceivable that the first trained function TF1 comprises a neural network. The first trained function TF1 may in this case be adapted as a function of a comparison of the second health information about the patient ascertained in the optional step T3 and the first health information about the patient, as well as the plurality of patient information PI. The first trained function TF1 may be adapted, as described above, in particular by means of a supervised learning, an unsupervised learning or a reinforcement learning process.

It is equally conceivable that the method for adapting the first trained function TF1 is performed on the inventive system SYS. In this case, step T1 of receiving the first trained function TF1 may be omitted. The training dataset may in this case be received in particular via the interface SYS.IF, the optional step of ascertaining the second health information about the patient and/or the step of adapting the second trained function being performed by means of the computing unit SYS.CU.

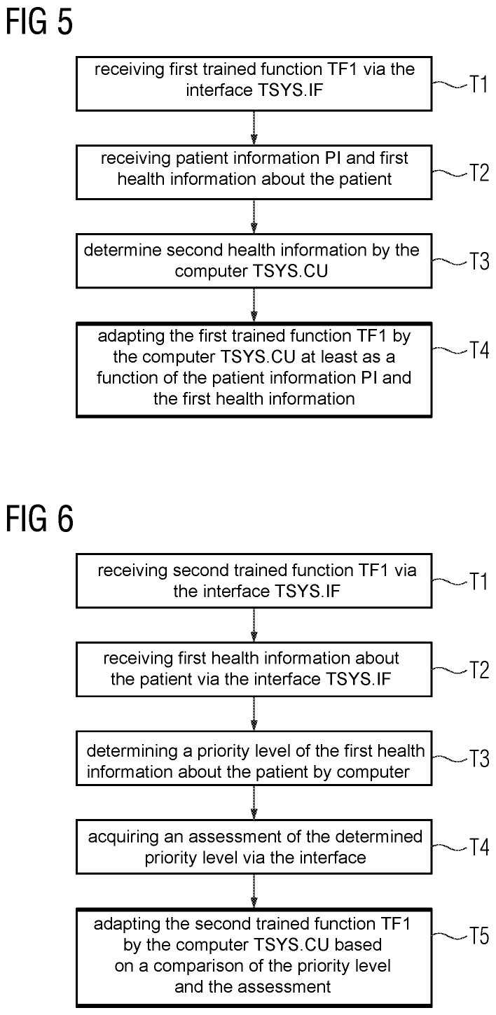

FIG. 6 shows a flowchart of an inventive method for adapting the second trained function TF2.

In step T1, the second trained function TF2 is received by means of the interface TSYS.IF. In this case the second trained function TF2 may be received for example from a system SYS or from a determination unit SYS.DU of a system SYS.

In step T2, the first health information about the patient is received by means of the interface TSYS.IF. The first health information about the patient may in this case represent a training dataset which can be read in from an internal data storage medium of the training system TSYS and/or from an external data storage medium.

In step T3, a priority level of the first health information about the patient is determined by means of the computing unit TSYS.CU as a function of the second trained function TF2. The second trained function TF2 may in this case comprise in particular an artificial neural network, a multilayer neural network and/or a convolutional neural network. Such a neural network may be configured to output the priority level of the first health information about the patient when the first health information about the patient is transmitted as an input vector to the second trained function TF2.

In step T4, an assessment in respect of the determined priority level of the first health information about the patient is acquired by means of the interface TSYS.IF. The assessment in respect of the determined priority level of the first health information about the patient may in this case be sourced from a training dataset which is correlated with the first health information about the patient. However, it is equally conceivable that the assessment in respect of the determined priority level of the first health information about the patient is input by a user by means of an input unit TSYS.OIFi of a user interface TSYS.OIF according to an above-described embodiment.

In step T5, the second trained function TF2 is adapted by means of the computing unit TSYS.CU as a function of a comparison of the priority level of the first health information about the patient and the assessment in respect of the determined priority level of the first health information about the patient. The second trained function TF2 may be adapted, as described above, in particular by means of a supervised learning, an unsupervised learning or a reinforcement learning process.

it is equally conceivable that the method for adapting the second trained function TF2 is performed on the inventive system SYS. In this case, step T1 of receiving the second trained function TF2 may be omitted. The training dataset may in this case be received in particular via the interface SYS.IF, the priority level of the first health information about the patient being determined and/or the second trained function TF2 being adapted by means of the computing unit SYS.CU.

It is furthermore conceivable that the system SYS and/or the training system TSYS are configured to adapt the first trained function TF1 and/or the second trained function TF2 according to an above-described embodiment.

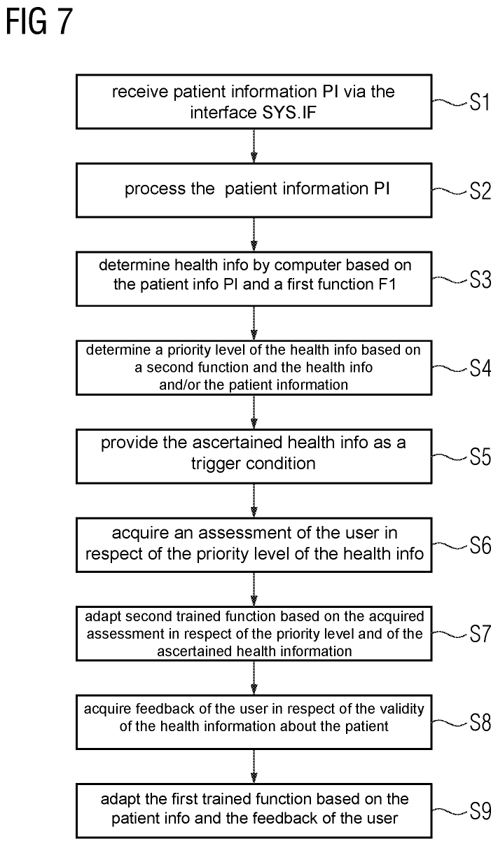

FIG. 7 shows a flowchart of an inventive method for providing information about a state of health of a patient.

In step S1, the plurality of patient information PI of the patient is received by means of the interface SYS.IF, the plurality of patient information PI including at least two different medical parameters assigned to the patient.

In an example, the method for monitoring the state of health of the patient is started automatically as soon as new patient information pertaining to a patient is received from an information source. However, the method may also be started by means of an input of the user and/or as a function of an arrival of a predetermined criterion.

In a further example, the receiving of the plurality of patient information PI comprises receiving first patient information from a first information source and receiving second patient information from a second information source which is different from the first information source. It is conceivable that the plurality of patient information PI of the patient is forwarded to the interface SYS.IF of the system SYS from a plurality of information sources, such as at least one digital patient health record and a smart device of the patient. However, the plurality of patient information PI may also be received from further information sources and/or other of the above-cited information sources. It is conceivable in particular that a part of the plurality of patient information PI, such as e.g. clinical findings and/or a description of symptoms of the patient, is present in an unstructured file format.

In the optional step S2, the plurality of patient information PI undergoes processing, comprising
quantifying the evolution over time of the at least one medical parameter, and/or
determining a normal value of the at least one medical parameter, further comprising
determining a deviation of the at least one medical parameter from the normal value,
wherein at least one piece of patient information of the received plurality of patient information PI comprises a pointer to an evolution over time of at least one medical parameter of the patient.

In an exemplary embodiment, the plurality of patient information PI is processed by means of the first function F1. For this purpose, the first function F1 may in particular include an algorithm and/or a model which is configured to process, correlate and reformat one or more medical parameters of the plurality of patient information PI, and/or to evaluate the same in a comparison with patient information of a reference patient as well as further information. The first function F1 may additionally include an image processing algorithm which is configured to segment images or image data of an imaging method and determine a dimension and/or volume of a physiological and/or pathological structure of the patient. The first function F1 may in this case be executed by means of the computing unit SYS.CU.

In an example, the quantification of the evolution over time of the at least one medical parameter comprises a comparison of the evolution of the at least one medical parameter with an evolution over time of a second medical parameter and/or of a limit value specified by experts. The at least one medical parameter in this case comprises for example a urine value, a blood value, a description of a symptom and/or a dimension of a pathological structure of the patient.

In a further example, the determining of the normal value of the at least one medical parameter comprises determining a limit below which 95% of all known measured values of the at least one medical parameter lie. Next, a deviation of a current measured value (which, for example, initializes the inventive method) of the at least one medical parameter from the normal value is determined in order to provide a reference base adapted to fit individual requirements of the patient.

In one embodiment, the plurality of patient information PI is processed as a function of a sensor data fusion method.

In an exemplary embodiment, the sensor data fusion method comprises a model and/or an algorithm which are configured to replace and/or correct missing and/or incorrect measured values of at least one parameter, to supplement implausible measured values as a function of other medical parameters of the patient and/or to generate virtual parameters. In a particularly simple example, the virtual parameter is a body mass index (BMI) which relates a weight of the patient to a square of a body height of the patient. The virtual parameter of the body mass index may therefore be referred to directly for a comparison with corresponding parameters of reference patients and/or limit values defined by expert communities. In a further example, the sensor data fusion method comprises determining the dimension of a tumor on the basis of images and/or image data of a plurality of imaging methods, such as e.g. magnetic resonance tomography, computed tomography and/or positron-emission tomography. Furthermore, the sensor data fusion method may also comprise determining measured values of medical parameters which are missing in a current set of the plurality of patient information PI. The determination of the measured values of the medical parameters may for example comprise an interpolation and/or extrapolation of the missing measured values as a function of existing medical parameters on the basis of earlier sets of the plurality of patient information PI of the patient. In addition, however, (simulation) models and/or empirical functions may also be used in the sensor data fusion method.

According to an embodiment, a part of the plurality of patient information PI is present in an unstructured file format, the processing of the plurality of patient information PI comprising extracting the part of the plurality of patient information PI into a structured file format by means of the computing unit SYS.CU, wherein the extracting of the part of the plurality of patient information PI is performed as a function of a computational linguistics method.

In an example, a part of the plurality of patient information PI is present in a text format, such as e.g. a .doc, an .rtf, a .txt, a .pdf, an .odt, an .htm, an .xls or a comparable file format. The part of the plurality of patient information may in this case comprise in particular clinical findings or a part of clinical findings pertaining to the patient. In an exemplary embodiment, a medical parameter is extracted from the part of the plurality of patient information PI by using a text mining method and/or by means of a multilayer neural network or a MultiNet. It is conceivable that the multilayer neural network is configured to conduct a semantic analysis of the part of the plurality of patient information PI. The multilayer neural network or MultiNet may be trained to interpret specific technical terms and/or technical jargon of clinical findings. It is further conceivable that a part of a pipeline model is used according to an above-described embodiment for processing the part of the plurality of patient information PI. In this case, a statistical model and/or a logical model may also be used in addition.

In an embodiment, the processing of the plurality of patient information comprises checking for the presence of at least one new piece of patient information and/or an appointment, the ascertaining of the health information about the patient and/or the providing of the health information about the patient being carried out as a function of the presence of the at least one new piece of patient information and/or of the appointment.

In step S3, the health information about the patient is ascertained by means of a computing unit SYS.CU as a function of the plurality of patient information PI and a first function F1 and a check is conducted, based on the ascertained health information about the patient, to determine whether a trigger condition is fulfilled.

The first function F1 may for example comprise an intelligent algorithm and/or a model. It is furthermore conceivable that the first function F1 is a first trained function TF1, the first trained function TF1 and/or the second trained function TF2 being based on an artificial neural network, a multilayer neural network, a convolutional neural network, a nearest-neighbor classification, a support vector machine and/or a Bayesian network. The computing unit SYS.CU is configured to execute the first trained function TF1 or, as the case may be, the first trained function TF1 and/or the second trained function TF2. The plurality of patient information PI may in this case represent input data or boundary conditions which are necessary for ascertaining the health information about the patient.

In an embodiment, the health information about the patient is ascertained as a function of the first function TF1 as well as of the evolution over time of the at least one medical parameter and/or the deviation of the at least one medical parameter from the normal value. It is conceivable that the evolution over time of the at least one medical parameter and/or the deviation of the at least one medical parameter from the normal value are determined by means of a separate function, such as e.g. a third function or a further function, of the system SYS. It is equally conceivable that the first function F1 is configured to determine the at least one medical parameter and/or the deviation of the at least one medical parameter from the normal value.

In an embodiment, ascertaining the health information about the patient comprises determining a parameter configuration from the plurality of patient information, and ascertaining the health information based on the determined parameter configuration.

In an example, the plurality of patient information may comprise a sleeping behavior of the patient. By applying the first function F1 to the plurality of patient information it is possible, inter alia, to determine a sleep duration and/or a duration of a slow-wave sleep phase of the patient, which, in particular in connection with further medical parameters of the patient, may be correlated with a nutritional condition, the state of health of and/or a mental capacity of the patient.

According to an embodiment, ascertaining the health information comprises correlating the determined parameter configuration with one or more reference parameter configurations, each of the reference parameter configurations indicating health information and an ascertaining of the health information based on the correlation step.

The reference parameter configuration may in this case be assigned to one or more reference patients that are assigned with regard to a medical condition to a critical group and/or to a non-critical group. The determined parameter configuration of the patient may be determined for example as a function of a deviation of a medical parameter as well as of medical parameters dependent thereon from a normal value, an expected evolution over time and/or a comparison with a corresponding parameter of one or more reference patients. The parameter configuration may in this case be determined in particular by means of the first function F1 and/or a third function. The correlation of the parameter configuration of the patient with the reference parameter configuration of one or more reference patients may in this case comprise in particular a normalization, a factorization, an interpolation, an extrapolation and/or a formation of an empirical model. In this way a (statistical) connection may be derived between the parameter configuration of the patient and a likelihood of a medical condition and/or a critical change in the state of health of the patient.

In a further embodiment, the ascertaining of the health information comprises correlating the determined parameter configuration with a comparison parameter configuration of a reference patient and ascertaining the health information based on the correlation step.

A check is carried out as a function of the ascertained health information about the patient to determine whether a trigger condition is fulfilled. The check may be conducted by means of the computing unit as a function of the first function, the second trained function and/or a third function. In an example, the trigger condition is fulfilled if an atypical configuration of medical parameters of the patient is present. In a further example, the trigger condition is not fulfilled if a physical constitution of the patient has remained unchanged in comparison with a most recently ascertained physical constitution.

In an embodiment, the check whether, based on the ascertained health information about the patient, a trigger condition is fulfilled, is conducted as a function of a determined priority level of the health information about the patient.

In an optional step S4, a priority level of the health information about the patient is determined by means of the computing unit SYS.CU as a function of a second function, as well as of the health information about the patient and/or of the plurality of patient information PI.

In an exemplary embodiment, the priority level constitutes a measure for a relevance of the ascertained health information about the patient. For example, a low priority level is determined if a markedly increased medical parameter of the patient compared to corresponding parameters of a plurality of reference patients still lies in a normal range. Conversely, a high priority level may be determined if a medical parameter of the patient deviates only slightly from the normal value of the patient but coincides with a value range of a parameter of a critical group of reference patients. The second function may in this case be in particular a second trained function TF2 which is based for example on an artificial neural network, a multilayer neural network and/or an expert system. The priority level of the health information about the patient is referred to in particular for checking whether the trigger condition is fulfilled.

In the further step S5, the ascertained health information about the patient is provided as a function of the trigger condition. In an example, a trigger condition is fulfilled because an atypical or unusual configuration of medical parameters has been identified in relation to the patient. The providing of the ascertained health information about the patient may in this case comprise a prioritizing of the patient in a worklist of the user, as well as an outputting of the ascertained health information about the patient to the user by means of the output unit SYS.OIFo of the user interface SYS.OIF, and a storing of the ascertained health information about the patient in a memory unit. It is furthermore conceivable that the ascertained health information about the patient is transferred to a medical device and/or to a private device of the patient. The output unit SYS.OIFo may be for example a screen, a monitor or a touchscreen which provides a visual output to the user. The private device of the patient may in particular be a smart device, such as e.g. a smartwatch, a smartphone or a tablet. In an embodiment, the providing of the health information comprises an output of a recommendation in respect of a performance of a medical test and/or a diagnostic method. According to embodiments, step S5 comprises a providing of control commands for controlling the diagnostic assessment station OP by means of the computing unit SYS.CU, the control commands being suitable for prioritizing the patient in a worklist of the user hosted in the diagnostic assessment station OP as a function of the ascertained health information, and/or for outputting the ascertained health information about the patient to the user and the control commands to the diagnostic assessment station OP by means of the computing unit SYS.CU. Further in step S5, the control commands may be forwarded to the diagnostic assessment station OP.

In a further example, the trigger condition is not fulfilled because the state of health of the patient is unchanged compared to a previously ascertained state of health. In this case the ascertained health information about the patient may be stored in the memory unit and the patient noted with a low priority in the worklist of the user. In an exemplary embodiment, the providing of the ascertained health information about the patient comprises at least a storing of the health information about the patient in a memory unit of a computer, a notebook, a server and/or a cloud.

In an embodiment, the trigger condition is considered fulfilled if a predetermined limit value for the priority level is exceeded. The providing of the ascertained health information about the patient may in this case comprise an outputting of the ascertained health information about the patient to the user. However, it is equally conceivable that the trigger condition remains unfulfilled on account of a low priority level of the health information. In this case the providing of the ascertained health information about the patient may comprise in particular a storing of the ascertained health information about the patient in a memory unit.

In an embodiment, the first function TF1 is further configured to determine an abnormality value for at least a part of the patient information PI, which abnormality value indicates the degree to which the patient information PI deviates from a norm. In the step of checking the trigger condition, the trigger condition is fulfilled if the abnormality value exceeds a predefined threshold.

In an embodiment, the providing of the ascertained health information about the patient comprises an output of the ascertained health information about the patient to the user as a function of the determined priority level of the health information about the patient.

The second function and/or the computing unit SYS.CU may in this act in particular as a decision-making entity which specifies whether the ascertained health information about the patient is output to the user. For example, the ascertained health information about the patient may be output to the user if a high priority level is present. Conversely, a corresponding output may be omitted if a low priority level is present. The priority level of the health information about the patient may in this case comprise in particular a predetermined value range. The value range may include a threshold value which, if exceeded, causes an output of the ascertained health information about the patient to be initiated.

According to a further embodiment, the second function is a second trained function TF2, wherein the providing of the ascertained health information about the patient comprises outputting the ascertained health information about the patient to the user. In this case the user may be invited in particular to make an assessment of the priority level of the health information about the patient.

The ascertained health information about the patient to the user as well as the invitation to the user to make the assessment of the priority level of the health information about the patient may be output to the user by means of the output unit SYS.OIFo. It is conceivable that the ascertained health information about the patient and the invitation to the user to make the assessment of the priority level of the health information about the patient are output in the form of a visual output, such as e.g. a graphical representation and/or a text-based description, but also an acoustic output, such as e.g. an alert tone and/or a voicemail. The invitation to the user to make the assessment of the priority level of the health information about the patient may in this case comprise in particular a query about whether the user considers the determined priority level of the health information about the patient appropriate. Such a query may be text-based, for example, by means of a dialog system or a chatbot, but also icon-based, by means of a selectable “thumbs-up” or “thumbs-down” element, as well as an assessment in the form of stars on a scale. In addition, other well-known assessment mechanisms are of course conceivable by means of which the user can make a time-efficient assessment of the determined priority level of the health information about the patient.

In a further embodiment, the providing of the ascertained health information about the patient comprises outputting the ascertained health information about the patient to the user, the first function F1 being a first trained function TF1. In this case the user may be invited in particular to provide feedback in respect of a validity of the health information about the patient.

As described above, the invitation to the user to provide feedback in respect of the validity of the health information about the patient may also comprise an output on the output unit SYS.OIFo of the user interface SYS.OIF. A corresponding output may in particular be a visual output and/or an acoustic output. In an exemplary embodiment, the output is structured in such a way that the user can carry out a time-efficient correction of the ascertained health information about the patient by means of the input unit SYS.OIFi. It is conceivable that the user is able to edit and/or amend the ascertained health information about the patient by means of the input unit SYS.OIFi. However, it is equally conceivable that the user may, as described above, input a text-based and/or icon-based assessment of the validity of the health information about the patient.

In an optional step S6, the assessment of the user in respect of the priority level of the health information about the patient is acquired by means of the interface SYS.IF.

It is conceivable that the assessment of the user in respect of the priority level of the health information about the patient is received by means of the input unit SYS.OIFi and forwarded to the interface SYS.IF. The interface SYS.IF may in this case represent in particular a communications interface which is configured to receive data from components of the system SYS and pass said data on to components of the system SYS. In this case the input unit SYS.OIFi of the user interface SYS.OIF may also be present mechanically separated from the system SYS and/or be connected to a mobile device, such as e.g. a notebook, a tablet or a smartphone. The assessment of the user in respect of the priority level of the health information about the patient may in this case be forwarded in particular wirelessly to the interface SYS.IF.

In a further optional step S7, the second trained function TF2 is adapted by means of the computing unit SYS.CU at least as a function of the acquired assessment of the user in respect of the priority level of the health information about the patient and of the ascertained health information about the patient.

In this embodiment, the second trained function TF2 may in particular comprise an artificial neural network or a multilayer neural network. For example, the second trained function TF2 comprises a multilayer neural network which is adapted by means of the computing unit SYS.CU on the basis of a backpropagation method. In an exemplary embodiment, such a multilayer neural network is configured to determine the priority level of the health information about the patient by means of the computing unit SYS.CU. A difference can subsequently be formed between the actual output (determined priority level of the health information about the patient) and the target output (assessment of the determined priority level of the first information about the state of health of the patient) of the multilayer neural network, which is regarded as an error. The error may subsequently be propagated back from an output layer to an input layer of the multilayer neural network. In this case a configuration of the multilayer neural network, in particular a weighting of connections between neurons, may be changed as a function of its effect on the error. The error between the target output and the actual output of the multilayer neural network can be minimized for an input pattern by means of corresponding methods. After being adapted, the adapted second trained function TF2 can be stored in a memory unit of the system SYS. It is equally conceivable that in order to be adapted, the second trained function TF2 is forwarded to a training system according to FIG. 3 and adapted as described above.

An optional step S8 comprises acquiring the feedback of the user in respect of the validity of the health information about the patient by means of the interface SYS.IF.

The feedback of the user in respect of the validity of the information about the state of health of the patient may, as described above, be received initially by means of the input unit SYS.OIFi of the user interface SYS.OIF of the system SYS and subsequently be transferred to the interface SYS.IF. The user interface SYS.OIF may in this case be present in particular mechanically separated from the system SYS and be connected to a mobile device. The mobile device may be configured to transfer the feedback of the user in respect of the validity of the information about the state of health of the patient wirelessly to the interface SYS.IF. The user interface SYS.OIF may, however, also be connected to the system SYS, as shown in FIG. 1.

A further optional step S9 comprises an adapting of the first trained function TF1 by means of the computing unit SYS.CU at least as a function of the plurality of patient information PI of the patient, as well as of the registered feedback of the user in respect of the validity of the information about the state of health of the patient.

For example, the feedback of the user in respect of the validity of the information about the state of health of the patient comprises a correction and/or an amendment of the information about the state of health of the patient. The first trained function TF1 may in particular comprise an artificial neural network, a multilayer neural network and/or a convolutional neural network. Such a neural network may be adapted by means of the computing unit SYS.CU within the scope of a supervised learning, an unsupervised learning or a reinforcement learning process. For this purpose, the information about the state of health of the patient may be ascertained initially by means of the first trained function TF1 on the basis of the plurality of patient information PI.

Following this, an adjustment of the configuration of the first trained function TF1 may be performed on the basis of a comparison of a target output (e.g. correction and/or amendment of the information about the state of health of the patient) and an actual output (ascertained information about the state of health of the patient) of the first trained function TF1 as a function of mathematical methods, such as e.g. a delta rule, a backpropagation method or an SGD method.

In an embodiment, a difference is formed between the actual output and the target output of the neural network, which difference is regarded as an error. The error may subsequently be propagated back from an output layer to an input layer of the multilayer neural network. In this case the configuration of the neural network, in particular a weighting of connections between neurons, may be changed as a function of its effect on the error. The error between the target output and the actual output of the multilayer neural network can be minimized for an input pattern by means of corresponding methods. After being adapted, the adapted first trained function TF1 can be stored in a memory unit of the system SYS. It is equally conceivable that in order to be adapted, the first trained function TF1 is forwarded to a training system according to FIG. 3 and adapted as described above.

In a further embodiment, the first trained function TF1 comprises a nearest-neighbor classification. The registered feedback of the user in respect of the validity of the information about the state of health of the patient may in this case represent in particular information desired by the user about the state of health of the patient in respect of the plurality of patient information PI. When the first trained function TF1 is adapted, the plurality of patient information PI as well as the difference feedback of the user in respect of the validity of the information about the state of health of the patient form a training dataset which is stored by means of the computing unit SYS.CU. The nearest-neighbor classification of the first trained function TF1 is therefore adapted to fit the information desired by the user about the state of health of the patient. It is equally conceivable that in order to be adapted, the first trained function TF1 is forwarded to a training system according to FIG. 3 and adapted as described above.

Where not yet explicitly realized, though beneficial and within the meaning of the disclosure, individual exemplary embodiments and individual subordinate aspects or features thereof may be combined with one another or interchanged without leaving the scope of the present disclosure. Advantages of the disclosure that are described with reference to one exemplary embodiment are also relevant, insofar as they are transferable, to other exemplary embodiments without being cited explicitly. In particular, the order of the method steps of the inventive method is to be understood as serving as an example. Individual steps may also be performed in a different order or may partially or completely overlap with respect to time.

To enable those skilled in the art to better understand the solution of the present disclosure, the technical solution in the embodiments of the present disclosure is described clearly and completely below in conjunction with the drawings in the embodiments of the present disclosure. Obviously, the embodiments described are only some, not all, of the embodiments of the present disclosure. All other embodiments obtained by those skilled in the art on the basis of the embodiments in the present disclosure without any creative effort should fall within the scope of protection of the present disclosure.

It should be noted that the terms “first”, “second”, etc. in the description, claims and abovementioned drawings of the present disclosure are used to distinguish between similar objects, but not necessarily used to describe a specific order or sequence. It should be understood that data used in this way can be interchanged as appropriate so that the embodiments of the present disclosure described here can be implemented in an order other than those shown or described here. In addition, the terms “comprise” and “have” and any variants thereof are intended to cover non-exclusive inclusion. For example, a process, method, system, product or equipment comprising a series of steps or modules or units is not necessarily limited to those steps or modules or units which are clearly listed, but may comprise other steps or modules or units which are not clearly listed or are intrinsic to such processes, methods, products or equipment.

References in the specification to “one embodiment,” “an embodiment,” “an exemplary embodiment,” etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described.

The exemplary embodiments described herein are provided for illustrative purposes, and are not limiting. Other exemplary embodiments are possible, and modifications may be made to the exemplary embodiments. Therefore, the specification is not meant to limit the disclosure. Rather, the scope of the disclosure is defined only in accordance with the following claims and their equivalents.

Embodiments may be implemented in hardware (e.g., circuits), firmware, software, or any combination thereof. Embodiments may also be implemented as instructions stored on a machine-readable medium, which may be read and executed by one or more processors. A machine-readable medium may include any mechanism for storing or transmitting information in a form readable by a machine (e.g., a computer). For example, a machine-readable medium may include read only memory (ROM); random access memory (RAM); magnetic disk storage media; optical storage media; flash memory devices; electrical, optical, acoustical or other forms of propagated signals (e.g., carrier waves, infrared signals, digital signals, etc.), and others. Further, firmware, software, routines, instructions may be described herein as performing certain actions. However, it should be appreciated that such descriptions are merely for convenience and that such actions in fact results from computing devices, processors, controllers, or other devices executing the firmware, software, routines, instructions, etc. Further, any of the implementation variations may be carried out by a general-purpose computer.

For the purposes of this discussion, the term “processing circuitry” shall be understood to be circuit(s) or processor(s), or a combination thereof. A circuit includes an analog circuit, a digital circuit, data processing circuit, other structural electronic hardware, or a combination thereof. A processor includes a microprocessor, a digital signal processor (DSP), central processor (CPU), application-specific instruction set processor (ASIP), graphics and/or image processor, multi-core processor, or other hardware processor. The processor may be “hard-coded” with instructions to perform corresponding function(s) according to aspects described herein. Alternatively, the processor may access an internal and/or external memory to retrieve instructions stored in the memory, which when executed by the processor, perform the corresponding function(s) associated with the processor, and/or one or more functions and/or operations related to the operation of a component having the processor included therein.

In one or more of the exemplary embodiments described herein, the memory is any well-known volatile and/or non-volatile memory, including, for example, read-only memory (ROM), random access memory (RAM), flash memory, a magnetic storage media, an optical disc, erasable programmable read only memory (EPROM), and programmable read only memory (PROM). The memory can be non-removable, removable, or a combination of both.

1. A computer-implemented method for controlling a diagnostic assessment station in a medical information network comprising a computing device and at least one diagnostic assessment station maintaining a data connection to the computing device and adapted to produce medical findings for a patient by a user, the method comprising:
receiving, at the computing device, patient information of the patient via an interface, wherein the patient information includes at least two different medical parameters assigned to the patient,
ascertaining health information about the patient by applying a first function hosted in the computing device to the patient information by the computing device,
checking, by the computing device, whether a trigger condition is fulfilled based on the ascertained health information about the patient,
providing, by the computing device, control commands for controlling the diagnostic assessment station based on the trigger condition, wherein the control commands are configured to prioritize the patient in a worklist of the user hosted in the diagnostic assessment station as a function of the ascertained health information, and/or to output the ascertained health information to the user via the diagnostic assessment station, and
outputting the control commands to the diagnostic assessment station by the computing device.

2. The method as claimed in claim 1, wherein the first function is configured to detect multivariate outliers in patient information.

3. The method as claimed in claim 2, wherein the first function for detecting multivariate outliers comprises a trained function that includes:
isolation forest algorithm,
elliptic envelope algorithm,
fast-minimum covariance determinant estimator (Fast MCD) algorithm, and/or
local outlier factors (LOF) algorithm.

4. The method as claimed in claim 1, wherein:
the first function is further configured to determine an abnormality value for at least a part of the patient information, and
in checking the trigger condition, the trigger condition is fulfilled if the abnormality value exceeds a predefined threshold.

5. The method as claimed in claim 4, wherein the health information is ascertained based on the abnormality value and/or the health information comprises the abnormality value.

6. The method as claimed in claim 4, wherein the control commands are configured to prioritize the patient in the worklist as a function of the abnormality value, wherein the patient is prioritized higher, the higher the abnormality value is.

7. The method as claimed in claim 4, wherein:
ascertaining the health information comprises determining a number of different abnormality values for the patient information by applying a number of different first functions for detecting multivariate outliers to the patient information by means of the computing unit, and
the health information is based on:
an aggregated abnormality value from the different abnormality values, and/or
an average abnormality value from the different abnormality values.

8. The method as claimed in claim 1, wherein the first function is configured to:
determine a predefined parameter configuration from the patient information, and
ascertain the health information based on a correlation of the determined parameter configuration with one or more predefined reference parameter configurations, wherein the reference parameter configurations indicate health information in each case.

9. The method as claimed in claim 8, wherein the ascertaining of the health information comprises:
a correlation of the determined parameter configuration with a comparison parameter configuration of a reference patient, and
an ascertaining of the health information based on the correlation.

10. The method as claimed in claim 1, wherein the health information comprises:
a diagnosis relating to the state of health of the patient,
a prognosis relating to the state of health of the patient,
a recommendation for action relating to the state of health of the patient, and/or
a health risk to the patient.

11. The method as claimed in claim 1, wherein the receiving of the plurality of patient information comprises:
receiving of first patient information from a first information source, and/or
receiving of second patient information from a second information source which is different from the first information source,
wherein the first information source and the second information source are configured as integrated into the medical information network and separate from the diagnostic assessment station.

12. The method as claimed in claim 1, wherein:
at least a piece of patient information of the received plurality of patient information comprises a pointer to an evolution over time of at least one medical parameter of the patient,
the method further comprises processing the plurality of patient information, including:
quantifying the evolution over time of the at least one medical parameter, and/or
determining a normal value of the at least one medical parameter,
a deviation of the at least one medical parameter from the normal value is determined in addition, and
the health information about the patient is ascertained as a function of the first function as well as of the evolution over time of the at least one medical parameter and/or of the deviation of the at least one medical parameter from the normal value.

13. The method as claimed in claim 1, further comprising:
providing patient information of a plurality of comparison patients in each case, wherein each comparison patient is associated with previously known health information, and
determining one or more reference patients from a plurality of comparison patients based on similarity measures, wherein one similarity measure is based on a similarity between the patient information of the patient and the patient information of the comparison patients,
wherein, in the step of ascertaining the health information, the health information is ascertained in addition based on the previously known health information of the reference patients.

14. The method as claimed in claim 1, comprising:
processing the plurality of patient information, including checking for the presence of at least one new piece of patient information and/or of an appointment,
wherein the health information about the patient is ascertained and/or the health information about the patient is provided as a function of the presence of the at least one new piece of patient information and/or of the appointment.

15. A computer program product comprising a computer program that is loadable directly into a memory of a system and includes program sections, that when executed by a processor of the system, causes the system to perform the method as claimed in claim 1.

16. A non-transitory computer-readable storage medium with an executable program stored thereon, that when executed, instructs a processor to perform the method of claim 1.

17. A system comprising:
an interface configured to receive patient information including at least two different medical parameters assigned to the patient; and
processing circuitry that is configured to:
apply a first function to the patient information to determine health information about a state of health of a patient based on the patient information,
check whether a trigger condition is fulfilled based on the ascertained health information about the patient,
determine control commands for controlling the diagnostic assessment station based on the trigger condition, wherein the control commands are configured to prioritize the patient in a worklist of the user hosted in the diagnostic assessment station as a function of the ascertained health information, and/or to output the ascertained health information to the user via the diagnostic assessment station, and
provide the determined control commands to the diagnostic assessment station maintaining a data connection with the system.
